(19) United States
(12) Patent Application Publication
(10) Pub. No.: US 2021/0022872 A1
(43) Pub. Date: Jan. 28, 2021

(54) BONE-DERIVED THERMOPLASTIC FILAMENT AND METHOD OF MANUFACTURE

(71) Applicants: David Louis Kirschman, Dayton, OH (US); Dewey J. Weeda, Oakwood, OH (US); Shawn A. Hunter, Springboro, OH (US)

(72) Inventors: David Louis Kirschman, Dayton, OH (US); Dewey J. Weeda, Oakwood, OH (US); Shawn A. Hunter, Springboro, OH (US)

(73) Assignee: D2 MEDICAL LLC, Dayton, OH (US)

(21) Appl. No.: 16/935,329

(22) Filed: Jul. 22, 2020

Related U.S. Application Data
(60) Provisional application No. 62/878,572, filed on Jul. 25, 2019.

(51) Int. Cl. A61F 2/30; A61L 27/36; A61L 27/44; A61L 27/54

(52) U.S. Cl. CPC A61F 2/30965; A61L 27/3608; A61L 27/44; A61L 27/54; A61F 2002/30065; A61L 2430/02; A61L 2430/38; A61F 2002/30957; A61F 2002/30968; A61L 27/3687

(57) ABSTRACT

A system, device/implant, method and processes for manufacturing a filament and an implant having at least one or a plurality of areas in the implant comprised of selectively-place bone to facilitate osteoconductivity and, potentially, osteoinductivity after the implant is implanted into a patient.

### CROSS-REFERENCE TO RELATED APPLICATION

The present application claims priority to provisional U.S. Application Ser. No. 62/878,572 filed Jul. 25, 2019. This provisional application is incorporated herein by reference and made a part hereof.

### BACKGROUND OF THE INVENTION

### 1. Field of the Invention

This invention relates to a system and processes for manufacturing a bone-thermoplastic filament for use in manufacturing an implant having at least one or a plurality of areas in the implant comprised of bone to facilitate osteoconductivity, and potential osteoinductivity, after the implant is implanted into a patient.

### 2. Description of the Related Art

In the past, medical implants were commonly used during surgical procedures. Human-derived bone allografts are commonly used in the treatment of orthopedic pathologies and injuries. Such grafts have the benefits of consolidating into host bone and promoting healing through bony fusion or arthrodesis. However, there are significant limitations to the application of natural bone allografts to such treatments.

Natural bone is available in limited anatomical shapes that may not be adequate for treatment of certain orthopedic pathologies. The ability to machine or form bone is limited for similar reasons. Recently, there have been advances in the use of three dimensional or volumetric methods for the manufacture of complex or customized medical devices. However, the heat required for manufacturing of raw material and final product limits the use of bioactive components into such devices.

Another common problem with allografts and implants of the past is the difficulty with which they can be manufactured with selective placement of bone in the implant or at predetermined or selected areas of the implant, such as bone-engaging surfaces of the implant. One problem with manufacturing implants or allografts is the difficulty of getting bone material strategically located in areas of the implant where it is desired to have improved osteoconductivity, and potentially osteoinductivity, after the implant is implanted into a patient.

In the past several years, considerable interest and development has occurred in computerized, three-dimensional printing techniques. For example, one process that is becoming more popular recently is the 3D printing technique used to manufacture models and prototypes. These techniques include ballistic particle manufacturing (BPM) and fusion deposition modeling (FDM), also known as fused filament fabrication (FFF). The process in general uses an ink-jet printing technique wherein an ink jet stream of liquid molten metal or metal composite material is used to create three-dimensional objects under computer control, similar to the way an ink jet printer produces two-dimensional graphic printing. A metal or metal composite part is produced by ink-jet printing of successive cross-sections, one layer after another to target using a cold welding (i.e., rapid solidification) technique, which causes bonding between particles in the successive layers.

Mammalian bone tissue is known to contain one or more proteinaceous materials, presumably active during growth and natural bone healing that can induce a developmental cascade of cellular events resulting in endochondral bone formation. The active factors are variously referred to in the literature as bone morphogenetic or morphogenic proteins (BMPs), bone inductive proteins, bone growth or growth factors, osteogenic proteins, or osteoinductive proteins. These active factors are collectively referred to herein as osteoinductive factors.

It is well known that bone contains these osteoinductive factors. These osteoinductive factors are present within the compound structure of cortical bone and are present at very low concentrations, e.g., 0.003%. Osteoinductive factors direct the differentiation of pluripotential mesenchymal cells into osteoprogenitor cells that form osteoblasts. Based upon the work of Marshall Urist as shown in U.S. Pat. No. 4,294,753, issued Oct. 13, 1981, proper demineralization of cortical bone exposes the osteoinductive factors, rendering it osteoinductive, as discussed more fully below.

The rapid and effective repair of bone defects caused by injury, disease, wounds, or surgery has long been a goal of orthopedic surgery. Toward this end, a number of compositions and materials have been used or proposed for use in the repair of bone defects. The biological, physical, and mechanical properties of the compositions and materials are among the major factors influencing their suitability and performance in various orthopedic applications.

Autologous cancellous bone (“ACB”) long has been considered the gold standard for bone grafts. ACB is osteoinductive and nonimmunogenic, and, by definition, it has all of the appropriate structural and functional characteristics appropriate for the particular recipient. Unfortunately, ACB is only available in a limited number of circumstances. Some individuals lack ACB of appropriate dimensions and quality for transplantation, and donor site pain and morbidity can pose serious problems for patients and their physicians.

Much effort has been invested in the identification and development of alternative bone graft materials. Various articles have been published on the theory of bone induction and a method for decalcifying bone, i.e., making demineralized bone matrix (DBM). DBM is an osteoinductive material, in that it induces bone growth when implanted in an ectopic site of a rodent, owing to the osteoinductive factors contained within the DBM. It is now known that there are numerous osteoinductive factors, e.g., BMP 1-15, which are part of the transforming growth factor-beta (TGF-beta) superfamily. BMP-2 has become the most important and widely studied of the BMP family of proteins. There are also other proteins present in DBM that are not osteoinductive alone but still contribute to bone growth, including fibroblast growth factor-2 (FGF-2), insulin-like growth factor-I and -II (IGF-I and IGF-II), platelet derived growth factor (PDGF), and transforming growth factor-beta 1 (TGF-beta 1).

DBM implants have been reported to be particularly useful (see, for example, U.S. Pat. Nos. 4,394,370, 4,440,750, 4,485,097, 4,678,470, and 4,743,259; each of which is incorporated herein by reference). DBM typically is derived from cadavers. The bone is removed aseptically and treated to kill any infectious agents. The bone is particulated by milling or grinding, and then the mineral component is extracted by various methods, such as by soaking the bone in an acidic solution. The remaining matrix is malleable and can be further processed and/or formed and shaped for implantation into a particular site in the recipient. Demineralized bone prepared in this manner contains a variety of components including proteins, glycoproteins, growth factors, and proteoglycans. Following implantation, the presence of DBM induces cellular recruitment to the site of injury. The recruited cells may eventually differentiate into bone forming cells. Such recruitment of cells leads to an increase in the rate of wound healing and, therefore, to faster recovery for the patient.

One problem with processing these implants was that subjecting them to heat can have adverse effects on proteins in the bone. Various articles have demonstrated that bone exposure to temperatures significantly exceeding physiologic ranges leads to denaturation of these key proteins and reduces or eliminates the implant's osteoinductive potential.

One important feature of an implant is the biomaterial property that the implant has to encourage osteoconduction, or the process by which bone grows on a surface. In general, it is preferred to have implants that are adapted to promote osteoconductivity in order to improve the chances that the implant will be well received by the patient and that the implant have characteristics that will promote osteoconduction.

The following references are noted and incorporated herein by reference and made a part hereof.

U.S. Pat. No. 5,490,962 appears to disclose solid free-form (SFF) techniques for making medical devices for controlled release of bioactive agent and implantation and growth of cells using computer aided design. Examples of SFF methods include stereo-lithography (SLA), selective laser sintering (SLS), ballistic particle manufacturing (BPM), fusion deposition modeling (FDM), and three dimensional printing (3DP). The macrostructure and porosity of the device can be manipulated by controlling printing parameters. Most importantly, these features can be designed and tailored using computer assisted design (CAD) for individual patients to optimize therapy.

U.S. Pat. No. 5,204,055 appears to disclose a process for making a component by depositing a first layer of a fluent porous material, such as a powder, in a confined region and then depositing a binder material to selected regions of the layer of powder material to produce a layer of bonded powder material at the selected regions. Such steps are repeated a selected number of times to produce successive layers of selected regions of bonded powder material so as to form the desired component. The unbonded powder material is then removed. In some cases the component may be further processed as, for example, by heating it to further strengthen the bonding thereof.

U.S. Pat. No. 5,518,680 appears to disclose solid free-form techniques for making medical devices for implantation and growth of cells from polymers or polymer/inorganic composites using computer aided design. Examples of SFF methods include stereo-lithography (SLA), selective laser sintering (SLS), ballistic particle manufacturing (BPM), fusion deposition modeling (FDM), and three dimensional printing (3DP). The devices can incorporate inorganic particles to improve the strength of the walls forming the pores within the matrix and to provide a source of mineral for the regenerating tissue. The devices can contain tissue adhesion peptides, or can be coated with materials which reduce tissue adhesion. The macrostructure and porosity of the device can be manipulated by controlling printing parameters. Most importantly, these features can be designed and tailored using computer assisted design (CAD) for individual patients to optimize therapy.

U.S. Pat. No. 6,783,712 appears to disclose a fiber-reinforced, polymeric implant material useful for tissue engineering, and method of making same. The fibers are preferably aligned predominantly parallel to each other, but may also be aligned in a single plane. The implant material comprises a polymeric matrix, preferably a biodegradable matrix, having fibers substantially uniformly distributed therein. In preferred embodiments, porous tissue scaffolds are provided which facilitate regeneration of load-bearing tissues such as articular cartilage and bone. Non-porous fiber-reinforced implant materials are also provided herein useful as permanent implants for load-bearing sites.

U.S. Pat. No. 6,974,862 appears to disclose malleable, biodegradable, fibrous compositions for application to a tissue site in order to promote or facilitate new tissue growth. One aspect of this invention is a fibrous component (e.g., collagen, chitosan, alginate, hyaluronic acid, poly-lactic acid, poly-capralactone, and polyurethane) that provides unique mechanical and physical properties. The invention may be created by providing a vessel containing a slurry, said slurry comprising a plurality of natural or synthetic polymer fibers and at least one suspension fluid, wherein the polymer fibers are substantially evenly dispersed and randomly oriented throughout the volume of the suspension fluid; applying a force, e.g., centrifugal, to said vessel containing said slurry, whereupon said force serves to cause said polymer fibers to migrate through the suspension fluid and a mass at a furthest extent of the vessel, forming a polymer material, with said polymer material comprising polymer fibers of sufficient length and sufficiently viscous, interlaced, or interlocked to retard dissociation of said polymer fibers.

U.S. Pat. No. 6,989,029 appears to disclose an implantable cage for holding tissue graft material. The cage includes a chamber configured and dimensioned to receive the tissue graft material and at least one side wall defining the chamber. In one embodiment, the cage is made of a biodegradable material. In another embodiment, the cage is made of a material that includes a bone-growth enhancer.

U.S. Pat. No. 6,993,406 appears to disclose a method for forming a three-dimensional, biocompatible, porous scaffold structure using a solid freeform fabrication technique (referred to herein as robocasting) that can be used as a medical implant into a living organism, such as a human or other mammal. Imaging technology and analysis is first used to determine the three-dimensional design required for the medical implant, such as a bone implant or graft, fashioned as a three-dimensional, biocompatible scaffold structure. The robocasting technique is used to either directly produce the three-dimensional, porous scaffold structure or to produce an over-sized three-dimensional, porous scaffold lattice which can be machined to produce the designed three-dimensional, porous scaffold structure for implantation.

U.S. Pat. No. 7,582,309 appears to disclose demineralized bone matrix fibers and a demineralized bone matrix composition. The demineralized bone matrix fibers have an average fiber length in the range from about 250 μm to about 2 mm and an aspect ratio of greater than about 4. The demineralized bone matrix composition includes demineralized bone matrix fibers and a biocompatible liquid in an amount to produce a coherent, formable mass. The formable mass retains its cohesiveness when immersed in a liquid. Methods for making the demineralized bone matrix fibers and composition are also provided.

U.S. Pat. No. 7,744,597 appears to disclose a fiber, preferably bone fiber that has a textured surface, which acts as an effective binding substrate for bone-forming cells and for the induction or promotion of new bone growth by bone-forming cells, which bind to the fiber. Methods of using the bone fibers to induce or promote new bone growth and bone material compositions comprising the bone fibers are also described.

U.S. Pat. No. 9,745,452 appears to disclose a 3D printer polymer filament that improves strength of a polymer resin and provides durability by using graphene coated metal nanoparticles and carbon nanotubes, and expresses a function of the graphene coated metal nanoparticles and the carbon nanotubes as a filler, and a manufacturing method thereof. Accordingly, according to the present invention, the 3D printer polymer filament and the manufacturing method includes mixing the graphene coated metal nanoparticles, the carbon nanotubes, and the polymer, using the manufactured mixture to form a filament through extrusion, and forming a 3D printed article by using the filament, thereby improving the strength and the durability by using the graphene coated metal nanoparticles and the carbon nanotubes.

U.S. Patent Publication No. 2007/0110820 appears to disclose an osteoinductive composition, corresponding osteoimplants, and methods for making the osteoinductive composition. The osteoinductive composition comprises osteoinductive factors, such as may be extracted from demineralized bone, and a carrier. The osteoinductive composition is prepared by providing demineralized bone, extracting osteoinductive factors from the demineralized bone, and adding the extracted osteoinductive factors to a carrier. Further additives such as bioactive agents may be added to the osteoinductive composition. The carrier and osteoinductive factors may form an osteogenic osteoimplant. The osteoimplant, when implanted in a mammalian body, can induce at the locus of the implant the full developmental cascade of endochondral bone formation including vascularization, mineralization, and bone marrow differentiation. Also, in some embodiments, the osteoinductive composition can be used as a delivery device to administer bioactive agents.

U.S. Patent Publication No. 2014/0161843 appears to disclose a macroporous 3-D tissue engineering scaffold that are manufactured by contacting an article comprising multiple distinct macroparticulate porogens distributed within a polymer scaffold, wherein the porogens are selectively and sequentially dissolvable by corresponding biocompatible stimuli.

U.S. Patent Publication No. 2014/0191439 appears to disclose a method and apparatus adapted for the free-form manufacture of complex systems using multiple three-dimensional (3D) printing techniques using multiple materials on a continuously rotating disk with a flat surface in combination with the continuous increasing of distance between the material(s) source(s) and the build surface so as to allow for the continuous feed manufacturing of 3D Objects and complex systems. The continuous rotation of the build platform in combination with the continuous z-axis motion of the build platform results in the deposit of a continuously forming helically shaped layer that folds back onto previously deposited sections of the helix and thereby forms a 3D object or system of objects.

U.S. Patent Publication No. 2015/0054195 appears to disclose a method for producing bone grafts using 3-D printing that is employed using a 3-D image of a graft location to produce a 3-D model of the graft. This is printed using a 3-D printer and an ink that produces a porous, biocompatible, biodegradable material that is conducive to osteoinduction. This is porous poly methyl methacrylate (PMMA) made osteoinductive by demineralized bone (DMB). The ink is provided as a precursor powder and liquid. The powder contains DMB, sucrose crystals and a polymerization initiator. The liquid contains methyl methacrylate (MMA). Optional compounds include antibiotics, radio-pacifiers, and compounds to increase biodegradability. Once mixed, the MMA polymerizes to PMMA. The ingredients are proportioned so that the ink is delivered through a 10 gauge print nozzle for about 10 minutes per batch. Once the graft is placed, natural bone gradually replaces the graft.

U.S. Patent Publication No. 2015/0183166 appears to disclose a three-dimensional printing system and equipment assembly for the manufacture of three-dimensionally printed articles is provided. The equipment assembly includes a three-dimensional printing build system, an optional liquid removal system and an optional harvester system. The build system includes a conveyor, plural build modules and at least one build station having a powder-layering system and a printing system. The equipment assembly can be used to manufacture pharmaceutical, medical, and non-pharmaceutical/non-medical objects. It can be used to prepare single or multiple articles.

U.S. Patent Publication No. 2015/0190547 appears to disclose an osteoinductive composition, corresponding osteoimplants, and methods for making the osteoinductive composition. The osteoinductive composition comprises osteoinductive factors, such as may be extracted from demineralized bone, and a carrier. The osteoinductive composition is prepared by providing demineralized bone, extracting osteoinductive factors from the demineralized bone, and adding the extracted osteoinductive factors to a carrier. Further additives such as bioactive agents may be added to the osteoinductive composition. The carrier and osteoinductive factors may form an osteogenic osteoimplant. The osteoimplant, when implanted in a mammalian body, can induce at the locus of the implant the full developmental cascade of endochondral bone formation including vascularization, mineralization, and bone marrow differentiation. Also, in some embodiments, the osteoinductive composition can be used as a delivery device to administer bioactive agents.

U.S. Patent Publication No. 2016/0038655 appears to disclose a method for manufacturing a bioactive implant that includes the steps of (a) forming a mixture of an bioactive agent and a setting agent capable of transitioning from a flowable state to a rigid state; (b) converting the mixture into a flowable state; and (c) transitioning the mixture into a solid state in a shape of the implant.

U.S. Patent Publication No. 2016/0136887 appears to relate to 3D printer inputs that includes filaments comprising separated layers or sections. These inputs particularly including filaments may be prepared by coextrusion, microlayer coextrusion or multicomponent/fractal coextrusion. These inputs and specifically filaments are represented to enable layering or combining different materials simultaneously through one or more nozzles during the so-called 3D printing process. These techniques are represented to facilitate smaller layer sizes (milli, micro, and nano) different layer configurations as well as the potential to incorporate materials that would otherwise not be usable in standard 3D printer methods.

U.S. Patent Publication No. 2016/0198576 appears to disclose a printed 3D functional part that includes a 3D structure comprising a structural material, and at least one functional electronic device is at least partially embedded in the 3D structure. The functional electronic device has a base secured against an interior surface of the 3D structure. One or more conductive filaments are at least partially embedded in the 3D structure and electrically connected to the at least one functional electronic device.

U.S. Patent Publication No. 2016/0297104 appears to relate to 3D printer inputs that include filaments comprising separated layers or sections. These inputs particularly including filaments may be prepared by coextrusion, microlayer coextrusion or multicomponent/fractal coextrusion. These inputs and specifically filaments enable layering or combining different materials simultaneously through one or more nozzles during the so-called 3D printing process. These techniques facilitate smaller layer sizes (milli, micro, and nano) different layer configurations as well as the potential to incorporate materials that would otherwise not be usable in standard 3D printer methods.

U.S. Patent Publication No. 2016/0318247 appears to disclose a device for making an implant having a hollow region, the device comprising a print surface rotatable in a clockwise and counterclockwise direction about an axis of rotation; a print head disposed adjacent to and substantially transverse to the print surface, the print head configured to apply material used to make the implant on at least a portion of the print surface or heat material disposed on at least a portion of the print surface used to make the implant; and a base disposed adjacent to the print head and contacting the print surface, the base configured to be movable in forward, backward and lateral directions relative to the print head to make the implant having the hollow region. Methods of using the device and are also disclosed.

U.S. Patent Publication No. 2017/0252967 appears to relate to 3D printer inputs that include filaments comprising separated layers or sections. These inputs particularly including filaments may be prepared by coextrusion, microlayer coextrusion or multicomponent/fractal coextrusion. These inputs and specifically filaments are represented are represented to ed to enable layering or combining different materials simultaneously through one or more nozzles during the so-called 3D printing process. These techniques are also represented to facilitate smaller layer sizes (milli, micro, and nano) different layer configurations as well as the potential to incorporate materials that would otherwise not be usable in standard 3D printer methods.

U.S. Patent Publication No. 2017/0281829 appears to disclose a biocompatible structure that includes one or more base structures for regeneration of different tissues. Each base structure includes alternately stacked polymer layers and spacer layers. The polymer layer includes a polymer and tissue forming nanoparticles. The polymer includes polyurethane. The tissue forming nanoparticles includes hydroxypatites (HAP) nanoparticles, polymeric nanoparticles, or nanofibers. The spacer layer includes bone particles, polymeric nanoparticles, or nanofibers. The weight percentage of tissue forming nanoparticles to the polymer in the polymer layer in one base structure is different from that in the other base structures. A method of producing the biocompatible structure includes forming multiple base structures stacked together, coating the stacked multiple base structures, and plasma treating the coated structure.

U.S. Patent Publication No. 2017/0296581 appears to disclose osteoinductive compositions and implants having increased biological activities, and methods for their production. The biological activities that may be increased include, but are not limited to, bone forming; bone healing; osteoinductive activity, osteogenic activity, chondrogenic activity, wound healing activity, neurogenic activity, contraction-inducing activity, mitosis-inducing activity, differentiation-inducing activity, chemotactic activity, angiogenic or vasculogenic activity, and exocytosis or endocytosis-inducing activity. In one embodiment, a method for producing an osteoinductive composition comprises providing partially demineralized bone, treating the partially demineralized bone to disrupt the collagen structure of the bone. In another embodiment, an implantable osteoinductive and osteoconductive composition comprises partially demineralized bone, wherein the collagen structure of the bone has been disrupted, and, optionally, a tissue-derived extract.

U.S. Patent Publication No. 2017/0362132 appears to disclose a method for producing a three-dimensional macroporous filament construct having interconnected microporous filaments showing a suitable surface roughness and microporosity. The method includes the steps of: a) preparing a suspension having particles of a predetermined material, a liquid solvent, one or more binders and optionally one or more dispersants, b) depositing the suspension in the form of filaments in a predetermined three-dimensional pattern, preferably in a non-solvent environment, thereby creating a three-dimensional filament-based porous structure, c) inducing phase inversion, whereby said filaments are transformed from a liquid to a solid state, by exposing the filaments during the deposition of the filaments with a non-solvent vapour and to a liquid non-solvent, d) thermally treating the structure of step d) by calcining and sintering the structure. A three-dimensional macroporous filament construct having interconnected microporous filaments showing a specific surface roughness and microporosity is shown. Various uses of the construct are shown, including its use for the manufacture of a biomedical product, such as a synthetic bone implant or bone graft.

What is needed, therefore, is an improved system, method and processes for manufacturing an implant that has improved osteoconductive capabilities and/or provides improved means for manufacturing an implant and selective placement of bone therein to promote osteoconduction.

### SUMMARY OF THE INVENTION

The purpose of this invention is to incorporate a bone allograft or xenograft into a thermoplastic filament that can be used for the manufacture of bioactive implants in a myriad of shapes and forms.

One object of the invention is to provide a system and process for manufacturing a filament having bone and thermoplastic in a predetermined ratio.

Another object of the invention is to provide a system, method and processes for controlling the distribution of bone in an implant.

Still another object of the invention is to provide manufacturing of a filament having bone and thermoplastic in a predetermined ratio that can then be used in a volumetric or 3D printing system.

Yet another object of the invention is to provide processing the filament to produce an implant having bone situated at areas in the implant where osteoconduction or improved osteoconduction is desired.

Another object of the invention is to provide a system, method and processes for mixing bone and thermoplastic in predetermined ratios, creating a filament using such mixture and then using such filament to produce an implant, such as by 3D-printing, injection molding or other types of manufacturing.

In another aspect, one embodiment of the invention provides a method of generating a bone-derived thermoplastic extrusion utilizing the mechanical combination of human or animal bone solid with at least one thermoplastic resin, such that there is uniform dispersal of the bone solid in the resin; the extrusion process comprising material pressure and heating upon a die, mold or runner to create a net shape; the extrusion comprising filament, pellet, bar, molding, three dimensional printing material stock, or similar structures; the bone proteins compromising collagen, bone morphogenetic proteins, osteocalcin, sialoprotein, osteopontin, osteonectin and other structural and functional proteins of bone.

In another aspect, one embodiment of the invention provides a bone-derived thermoplastic extrusion comprising a solid derived from human or animal bone; the bone combined with a thermoplastic resin such that there is uniform dispersal of the bone solid in the resin; the extrusion comprising filament, pellet, bar, molding, three dimensional printing material, or similar structures.

In another aspect, one embodiment of the invention provides an osteoconductive surgical implant manufactured from a bone-derived thermoplastic extrusion; the surgical implant incorporating a combination of human or animal bone-derived solid and thermoplastic with dispersal of the bone in the thermoplastic.

In another aspect, one embodiment of the invention provides a bone-derived thermoplastic filament comprising a human bone allograft, the bone allograft comprising mineral component and heat-resistant protein component, combined with a thermoplastic resin such that there is even dispersal of the bone allograft in the resin, heated and extruded to filament or pellet form; the bone allograft comprising a proteinaceous component; the proteinaceous component comprising mineralized collagen or other heat-resistant proteins; the thermoplastic resin comprising nylon, nylon, acrylonitrile butadiene styrene (ABS), polycarbonate, polyetherimide, polymethylmethacrylate (PMMA), acrylic, polyacryletherketones or similar biocompatible thermoplastic; the bone allograft comprising cortical bone powder, granule or fiber; the mixture of thermoplastic and bone allograft being a molded from or extrusion into a filament or pellet; the filament or pellet containing a minimum of 1% bone allograft by weight; the bone allograft form having a diameter no greater than 70% of the filament or pellet diameter; the filament being substantially flexible, such that it can be rolled onto a spool for shipping, handling and/or further manufacture; the filament adapted for the manufacture of medical devices using volumetric manufacturing methods, such as three dimensional printing; the filament, pellet and/or filament spool undergoing a terminal sterilization and packaging process via irradiation, heat or chemical means; the filament, incorporated into a medical device using volumetric manufacturing process, such as three dimensional printing.

In another aspect, one embodiment of the invention provides a bone-derived thermoplastic filament comprising a human bone allograft, the bone allograft comprising a mineral component combined with a thermoplastic resin such that there is even dispersal of the bone allograft in the resin, heated and extruded to filament or pellet form; the thermoplastic resin comprising nylon, acrylonitrile butadiene styrene (ABS), polycarbonate, polyetherimide, polymethylmethacrylate (PMMA), acrylic, polyacryletherketones or similar biocompatible thermoplastic; the bone allograft comprising cortical bone powder, granule or fiber; the mixture of thermoplastic and bone allograft being a molded from or extrusion into a filament or pellet; the filament or pellet containing a minimum of 1% bone allograft by weight; the bone allograft form having a diameter no greater than 70% of the filament or pellet diameter; the filament being substantially flexible, such that it can be rolled onto a spool for shipping, handling and/or further manufacture; the filament adapted for the manufacture of medical devices using volumetric manufacturing methods, such as three dimensional printing; the filament, pellet and/or filament spool undergoing a terminal sterilization and packaging process via irradiation, heat or chemical means; the filament, incorporated into a medical device using volumetric manufacturing process, such as three dimensional printing.

In another aspect, one embodiment of the invention provides a method of generating a thermoplastic filament or pellet by the following means the bone allograft mechanically processed to create powdered, granular, elongate, or fiber form; mixing the bone allograft with a thermoplastic resin, in a liquid or allograft process, such that there is even dispersal of the bone allograft in the resin; the mixing of the bone allograft with thermoplastic resin in proportions which maximize the proportion of bone by weight, while maintaining adequate mechanical properties of the resulting biomaterial; the mixture of thermoplastic and bone allograft being heated to create a liquefied composite, the composite being pressurized and formed through a die, mold, or similar means to create the filament or pellet; the mixing occurs in a heated state, with temperatures in excess of the melting point of the thermoplastic; the mixing comprising impeller agitation or ultrasonic agitation or other means; the mixing in a cool allograft state, where bone derived allograft is mixed with the thermoplastic below melting temperature of the thermoplastic; the mixing in a solid state comprising thermoplastic granules and bone derived allograft granules of substantially similar size and surface volume; the mixing in a solid state comprising physical agitation, ultrasonic means, to create an even dispersal of bone and thermoplastic allografts; the method performed in a substantially sterile environment, such as a clean room; the filament, pellet and/or filament spool undergoing a terminal sterilization and packaging process via irradiation, heat or chemical means; the filament and/or pellet incorporated into a three dimensional manufacturing process.

In another aspect, one embodiment of the invention provides a filament adapted for use in a volumetric or 3D printer or mold, the filament comprising: a thermoplastic of a first predetermined quantity; and processed bone of a second predetermined quantity; the first and second predetermined quantities being selected to define a desired ratio of bone to thermoplastic in response to a desired amount of bone in an implant manufactured using the filament.

In another aspect, one embodiment of the invention provides a system for making an implant having osteoconductive properties; the system comprising: a production station, the production station comprising at least one of volumetric printing, injection molding, machining, sintering, or forming device adapted to use a filament comprising a bone component and a thermoplastic component in a predetermined ratio; wherein the implant comprises exposed bone in predetermined areas of the implant to improve osteoconductivity after the implant is implanted into a patient.

In another aspect, one embodiment of the invention provides a method for making an osteoconductive implant having osteoconductive areas; the method comprising the steps of providing a filament comprising bone and thermoplastic in a predetermined ratio, the bone being substantially evenly dispersed in the thermoplastic in at least a portion of the filament; using the filament to produce the implant such that bone is located at the osteoconductive areas of the implant.

In another aspect, one embodiment of the invention provides a system for creating a filament having a mixture of bone and thermoplastic in a predetermined ratio, the system comprising a filament producing station for producing the filament, the station comprising an extruder adapted to receive the mixture and for creating the filament having the bone distributed substantially evenly in the thermoplastic and for extruding and producing the filament in response thereto.

This invention, including all embodiments shown and described herein, could be used alone or together and/or in combination with one or more of the features covered by one or more of the following list of features:

The method wherein bone is mixed with thermoplastic pellet in the solid state, undergoing mechanical agitation prior to or during the extrusion process; the mixing with the thermoplastic below the glass transition temperature of the thermoplastic; the mixing further comprising physical agitation, electrostatic adhesion, or ultrasonic means to create a uniform dispersal of bone and thermoplastic solids.

The method wherein bone is combined with thermoplastic solid and agitated within an extrusion chamber subjected to heat, and/or pressure by auger screw or similar means to create dispersal of the bone solid in the forming extrusion.

The method wherein bone is mixed with thermoplastic pellet in the liquid state, undergoing mechanical agitation prior to or during the extrusion process.

The method wherein bone solid is combined with heated thermoplastic liquid and mechanically mixing to create uniform dispersal prior to being placed in an extrusion chamber for extrusion process; the mixing comprising impeller agitation, ultrasonic agitation or other mechanical means resulting in a heated liquid state, with temperatures above the melting point of the thermoplastic, where the bone is added during and/or prior to the agitation and/or heating.

The method wherein the bone comprises mineral bone solid derived from human or animal bone, the bone treated via thermal, mechanical, or chemical processes to remove blood and lipids to reduce bioburden, leaving solid mineral components.

The method wherein the mineral components provide thermal stabilization to bone proteins, allowing for the proteins to avoid denaturation during extrusion heating.

The method wherein the bone is mechanically processed to create powdered, granular, elongate, or fiber form, with powder or granular forms having particles less than 1,000 μm in size, residual moisture content less than 6% by weight.

The method wherein the bone is mixed with thermoplastic resin in a specific ratio, the ratio is determined by mass, where the mass of thermoplastic resin ranges from 2 to 100 times the mass of the bone.

The method wherein the heating is applied for a short duration of time as to minimize thermal exposure to the bone solid.

The method wherein air or other gas is injected into the thermoplastic mixture during a preparation, heating, mixing or extrusion process to create a porous structure upon cooling.

The method wherein the filament is substantially flexible, such that it can be rolled onto a spool for handling and storage.

The method wherein the extrusion undergoes terminal sterilization via irradiation, heat or chemical means.

The extrusion wherein the bone comprises cortical bone powder, granule or fiber and is treated via thermal, mechanical, or chemical processes to remove blood and lipids and reduce bioburden, leaving solid mineral components.

The extrusion wherein the thermoplastic resin comprising nylon, acrylonitrile butadiene styrene (ABS), polycarbonate, polyetherimide, polymethylmethacrylate (PMMA), acrylic, polyacryletherketones or similar biocompatible thermoplastic.

The extrusion wherein the extrusion contains a minimum of 1% bone solid by weight.

The extrusion wherein the extrusion comprises a filament being substantially flexible, such that it can be rolled onto a spool for handling and/or optimized for use with volumetric manufacturing methods.

The extrusion wherein the extrusion undergoes terminal sterilization via irradiation, heat or chemical means.

The surgical implant manufactured utilizing volumetric printing, injection molding, machining, sintering, forming or similar means.

The surgical implant wherein there is substantially uniform dispersal of the bone component within the thermoplastic component.

The surgical implant wherein at least a portion of the bone-derived solid is exposed at the surface of the implant; the exposed bone-derived solid expressing osteoconductive and/or osteoinductive properties and imparting the properties to the implant.

The surgical implant of claim 23 wherein the bone-derived solid on specific surfaces exposed in a controlled manner by mechanical or chemical means for exposure of osteoconductive or osteoinductive elements where biologic response is desired; the chemical means comprising treatment of bone with acid such as acetic acid, citric acid, ethylenediamine tetraacetic acid, or hydrochloric acid.

The surgical implant wherein the implant comprises hygroscopic properties allowing for cellular and/or chemical diffusion and/or communication between internal bone-derived solids and the external implant surface.

The surgical implant wherein the implant is process-strengthened utilizing strain hardening, compression annealing, cross-linking, addition of strengthening additive, or similar means in order to accommodate physiological loading without failure.

The surgical implant wherein the implant possesses variable zones of differing bone content to impart regional mechanical and biological functions such as a diffusion gradient for directed biologic response.

The filament wherein the processed bone is at least one of sterilized or processed to reduce bioburden in the processed bone before it is added to the thermoplastic.

The filament wherein the processed bone is distributed substantially evenly with the thermoplastic in predetermined areas of the filament.

The filament wherein the processed bone is distributed substantially evenly with the thermoplastic substantially throughout the filament.

The filament wherein the processed bone has a particle size of less than 1,000 μm.

The filament wherein a mass of the thermoplastic is approximately two times a mass of the processed bone in the filament.

The filament wherein the processed bone comprises mineral bone solid derived from human or animal bone, the processed bone treated via thermal, mechanical, or chemical processes to remove blood and lipids to reduce bioburden, leaving solid mineral components.

The filament wherein the solid mineral components provide thermal stabilization to bone proteins, allowing for the bone proteins to avoid denaturation during heating.

The filament wherein the processed bone is mechanically processed to create powdered, granular, elongate, or fiber form, with powder or granular forms having particles less than 1,000 μm in size, residual moisture content less than 6% by weight.

The filament wherein the processed bone is mixed with the thermoplastic in a specific ratio, the ratio is determined by mass, where the mass of the thermoplastic ranges from 2 to 100 times the mass of the processed bone.

The filament wherein the processed bone is mixed with the thermoplastic in a specific ratio, the ratio is determined by mass, where the mass of the thermoplastic ranges from 10 to 50 times the mass of the processed bone.

The filament wherein the thermoplastic comprises nylon, acrylonitrile butadiene styrene (ABS), polycarbonate, polyetherimide, polymethylmethacrylate (PMMA), acrylic, polyacryletherketones or similar biocompatible thermoplastic.

The filament wherein the filament contains a minimum of 1% bone solid by weight.

The filament wherein the bone comprises cortical bone powder, granule or fiber.

The system wherein the bone component is substantially evenly distributed in the thermoplastic component in the filament before the filament is used to produce the implant.

The system wherein the bone component is at least one of sterilized or processed to reduce bioburden in the bone component before it is added to the thermoplastic component.

The system wherein the bone component is distributed substantially evenly with the thermoplastic component in predetermined areas of the filament.

The system wherein the bone component is distributed substantially evenly with the thermoplastic component substantially throughout the filament.

The system wherein the bone component has a particle size of between less than about 1,000 μm.

The system wherein the bone component has a particle size of less than about 500 μm.

The system wherein the predetermined ratio is on the order of the thermoplastic component being approximately two times a mass of the bone component.

The system wherein the bone component comprises mineral bone solid derived from human or animal bone, the bone component treated via thermal, mechanical, or chemical processes to remove blood and lipids to reduce bioburden, leaving solid mineral components.

The system wherein the solid mineral components provide thermal stabilization to bone proteins, allowing for the bone proteins to avoid denaturation during extrusion heating.

The system wherein the bone component is mechanically processed to create powdered, granular, elongate, or fiber form, with powder or granular forms having particles less than about 1,000 μm in size and a residual moisture content of less than 6% by weight.

The system wherein the bone component is mixed with the thermoplastic component in a specific ratio, the specific ratio is determined by mass, where the mass of the thermoplastic component ranges from 10 to 50 times the mass of the bone component.

The system wherein the bone component is mixed with the thermoplastic component in a specific ratio, the ratio is determined by mass, where the mass of the thermoplastic ranges from 2 to 100 times the mass of the bone component.

The system wherein the bone component comprises cortical bone powder, granule or fiber and is treated via thermal, mechanical, or chemical processes to remove blood and lipids and reduce bioburden, leaving solid mineral components.

The system wherein the thermoplastic component comprises nylon, acrylonitrile butadiene styrene (ABS), polycarbonate, polyetherimide, polymethylmethacrylate (PMMA), acrylic, polyacryletherketones or similar biocompatible thermoplastic.

The system wherein the extrusion contains a minimum of 1% bone solid by weight.

The system wherein the implant is manufactured from a bone-derived thermoplastic extrusion; the implant incorporating a combination of human or animal bone-derived solid and thermoplastic with dispersal of the human or animal bone-derived solid in the thermoplastic.

The system wherein the implant is manufactured utilizing volumetric printing, injection molding, machining, sintering, forming or similar means.

The system wherein there is substantially uniform dispersal of the bone component within the thermoplastic component.

The system wherein at least a portion of the human or animal bone-derived solid is exposed at the surface of the implant; the exposed bone-derived solid expressing osteoconductive and/or osteoinductive properties and imparting the properties to the implant.

The system wherein the exposed bone-derived solid on specific surfaces is deposited in a controlled manner by mechanical or chemical means for exposure of osteoconductive or osteoinductive elements where biologic response is desired; the chemical means comprising treatment of bone with acid such as acetic acid, citric acid, ethylenediamine tetraacetic acid, or hydrochloric acid.

The system wherein the implant comprises hygroscopic properties allowing for cellular and/or chemical diffusion and/or communication between internal bone-derived solids and an external surface of the implant.

The system wherein the implant is process-strengthened utilizing strain hardening, compression annealing, cross-linking, addition of strengthening additive, or similar means in order to accommodate physiological loading without failure.

The system wherein the implant possesses variable zones of differing bone content to impart regional mechanical and biological functions such as a diffusion gradient for directed biologic response.

The system wherein the system further comprises a filament production station for producing at least one filament; the filament production station comprising: an extruder having a feed hopper, the hopper being adapted to receive a mixture of bone and thermoplastic in a predetermined ratio, the extruder plasticating the mixture such that the bone is dispersed substantially evenly throughout the thermoplastic, thereby providing the filament for use at the production station.

The system wherein the system further comprises a mixing station for producing the mixture of bone and thermoplastic in the predetermined ratio.

The system wherein the predetermined ratio of the thermoplastic component is between two to one-hundred times the mass of the bone component.

The system wherein the bone comprises a particle size of less than about 1000 μm.

The system wherein the production station comprises at least one volumetric or 3D printer.

The system wherein the predetermined ratio is selected in response to osteoconductive properties of the implant.

The system wherein the at least one volumetric or 3D printer has a plurality of print heads, each of which is adapted to receive a filament having predetermined bone to thermoplastic ratio.

The system wherein the implant comprises predefined areas where osteoconductivity is desired, the at least one filament having bone and thermoplastic ratio such that when the printer prints the implant, the bone is located at the predefined areas.

The system wherein a plurality of filaments are used with the plurality of print heads, respectively, each of the plurality of filaments have a different bone to thermoplastic ratio, so that predefined areas of the implant also have corresponding different bone to thermoplastic ratio.

The system wherein the at least one filament is used in the print head and the implant comprises predefined areas where osteoconductivity is desired, the at least one filament having bone and thermoplastic ratio such that when the print head prints the implant and directs the bone to the predefined areas.

The method wherein the using step comprises the step of using a volumetric/3D printer or injection mold to print or mold, respectively the implant using the filament.

The method wherein the bone in the filament has a bone particle size of less than about 500 micrometers.

The method wherein the method further comprises the step of using a filament wherein the predetermined ratio of thermoplastic to bone is selected in response to the osteoconductive properties desired in the implant.

The method wherein the predetermined ratio of thermoplastic mass is approximately two times the mass of the bone.

The method wherein the predetermined ratio of thermoplastic mass is approximately ten times the mass of the bone.

The method wherein the predetermined ratio of thermoplastic mass is approximately fifty times the mass of the bone.

The method wherein the predetermined ratio of thermoplastic mass is approximately one hundred times the mass of the bone.

The method wherein the method further comprises the steps of determining an amount of bone to situate at the osteoconductive areas; using at least one volumetric/3D printer and the filament to situate at least some of the bone in the filament at the osteoconductive areas.

The method wherein the at least one volumetric/3D printer comprises a plurality of print heads, the method comprising the steps of using a first filament having a first predetermined ratio of bone to thermoplastic in one of the plurality of print heads; using a second filament having a second predetermined ratio of bone to thermoplastic in another of the plurality of print heads; wherein the first and second predetermined ratios are different.

The method wherein the method further comprises the steps of using a first filament having a first predetermined ratio of bone to thermoplastic in the at least one volumetric/3D printer to print a first portion of the implant; using a second filament having a second predetermined ratio of bone to thermoplastic in the at least one volumetric/3D printer to print a second portion of the implant; wherein the first and second predetermined ratios are different.

The method wherein the method further comprises the step of demineralizing the implant after it is produced in order for the bone to provide thermal protection to osteoinductive bone proteins, thereby avoiding protein denaturation during heating.

The method wherein the method further comprises the step of selecting a filament that will cause at least a portion of the osteoconductive areas to have a higher bone content than other portions of the implant.

The method wherein the method further comprises the step of selecting a filament that will cause at least a portion of the osteoconductive areas to have a low bone content than other portions of the implant.

The method wherein the method further comprises the step of processing the implant to increase a porosity of the implant to facilitate absorbing fluid having nutrients and/or cells that facilitate a healing response.

The method wherein the method further comprises the step of processing the bone to a predetermined particle size to provide processed bone; combining a predetermined amount of the processed bone with a predetermined amount of thermoplastic in the predetermined ratio to provide a mixture; feeding the mixture into an extruder; forming the filament using the extruder; using the filament during the using step.

The method wherein the implant is processed chemically or mechanically to expose the bone to facilitate osteoconduction.

A surgical implant for implanting into a person, the surgical implant being manufactured according to the method.

The system wherein the bone is distributed substantially evenly with the thermoplastic substantially throughout the filament.

The system wherein the bone has a particle size of 1,000 μm or less.

The system wherein the predetermined ratio is on the order of the thermoplastic being approximately two times to 100 times a mass of the bone.

The system wherein the bone is at least one of sterilized or processed to reduce bioburden in the bone before it is added to the thermoplastic.

The system wherein the bone is distributed substantially evenly with the thermoplastic in predetermined areas of the filament.

The system wherein the bone is distributed substantially evenly with the thermoplastic substantially throughout the filament.

The system wherein the bone has a particle size of less than 1,000 μm.

The system wherein the ratio is on the order of the thermoplastic being approximately two times to a hundred times a mass of the bone.

The system wherein the bone comprises mineral bone solid derived from human or animal bone, the bone treated via thermal, mechanical, or chemical processes to remove blood and lipids to reduce bioburden, leaving solid mineral components.

The system wherein the solid mineral components provide thermal stabilization to bone proteins, allowing for the bone proteins to avoid denaturation during extrusion heating.

The system wherein the bone is mechanically processed to create powdered, granular, elongate, or fiber form, with powder or granular forms having particles less than 1,000 μm in size, residual moisture content less than 6% by weight.

The system wherein the bone is mixed with the thermoplastic in a specific ratio, the ratio is determined by mass, where the mass of the thermoplastic ranges from 2 to 100 times the mass of the bone.

The system wherein the bone comprises cortical bone powder, granule or fiber and is treated via thermal, mechanical, or chemical processes to remove blood and lipids and reduce bioburden, leaving solid mineral components.

The system wherein the thermoplastic comprises nylon, acrylonitrile butadiene styrene (ABS), polycarbonate, polyetherimide, polymethylmethacrylate (PMMA), acrylic, polyacryletherketones or similar biocompatible thermoplastic.

The system wherein the extrusion contains a minimum of 1% bone solid by weight.

These and other objects and advantages of the invention will be apparent from the following description, the accompanying drawings and the appended claims.

### BRIEF DESCRIPTION OF THE ACCOMPANYING DRAWINGS

FIG. 1 is a schematic view of a system having a plurality of stations in accordance with one embodiment of the invention;

FIG. 2A is a view of a mixer for mixing thermoplastic and bone in predetermined ratios and also shows an extruder producing a filament in accordance with one embodiment of the invention;

FIGS. 2B1 and 2B2 are cross-sectional views of two different embodiments of a filament;

FIG. 2C is schematic showing a volumetric or 3D printer that is used at the implant device production station shown in FIG. 1;

FIG. 3 is a schematic view of the process steps occurring at the bone/thermoplastic processing and mixing station 12;

FIG. 4 is another schematic diagram illustrating various process steps relating to the implant/device production station 24 shown in FIG. 1;

FIG. 5A is a schematic diagram of a first illustrative process in accordance with one embodiment of the invention;

FIG. 5B is another schematic diagram of a second illustrative process in accordance with another embodiment of the invention;

FIGS. 6A-6D illustrate an implant produced in accordance with one embodiment of the invention and modified with bone component that is evenly dispersed in the polymer and throughout the implant;

FIGS. 7A-7D illustrate an implant produced in accordance with another embodiment of the invention showing the implant with a bone component selectively located near an external surface of the implant in order to increase osteoconduction; and

FIGS. 8A-8D illustrate an implant produced in accordance with another embodiment of the invention illustrating the implant with a bone component selectively located near internal or external surfaces for increased mechanical strength and improved osteoconduction.

### DETAILED DESCRIPTION OF THE PREFERRED EMBODIMENTS

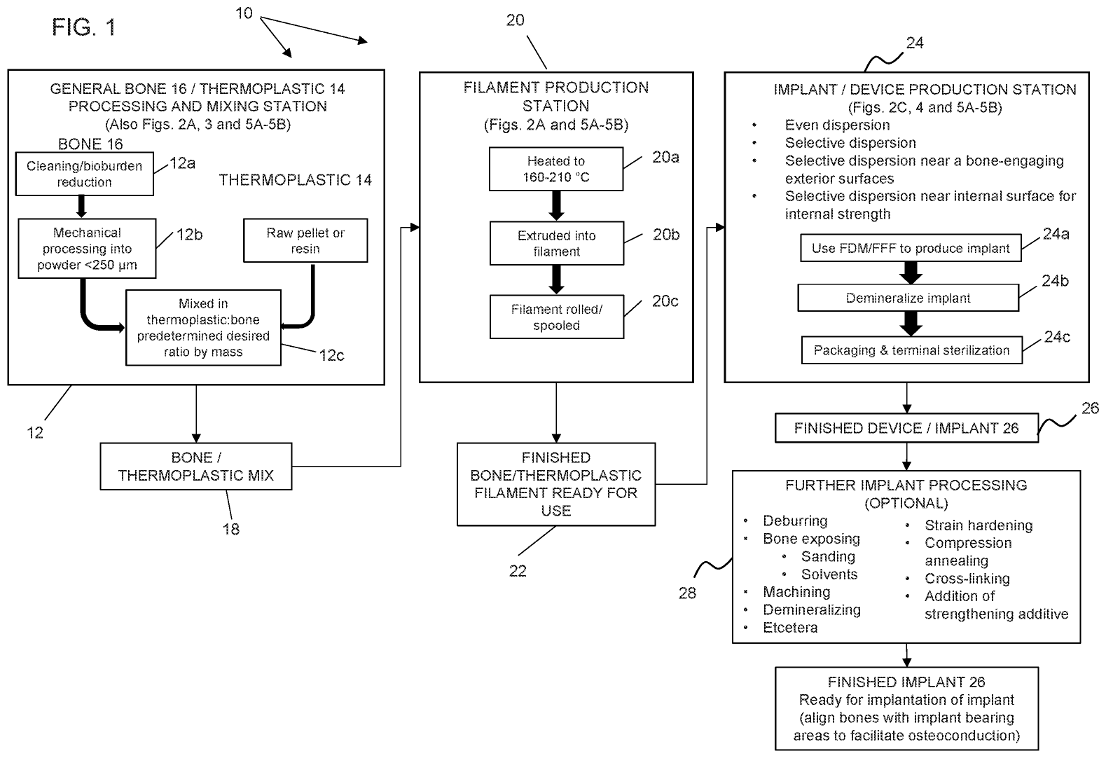

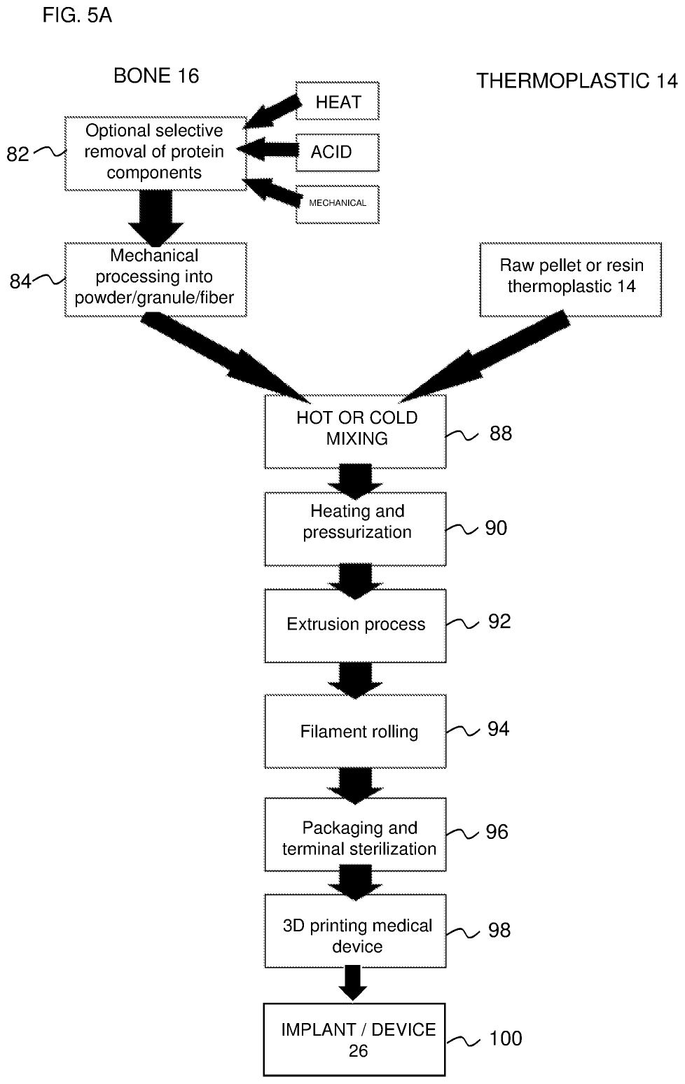

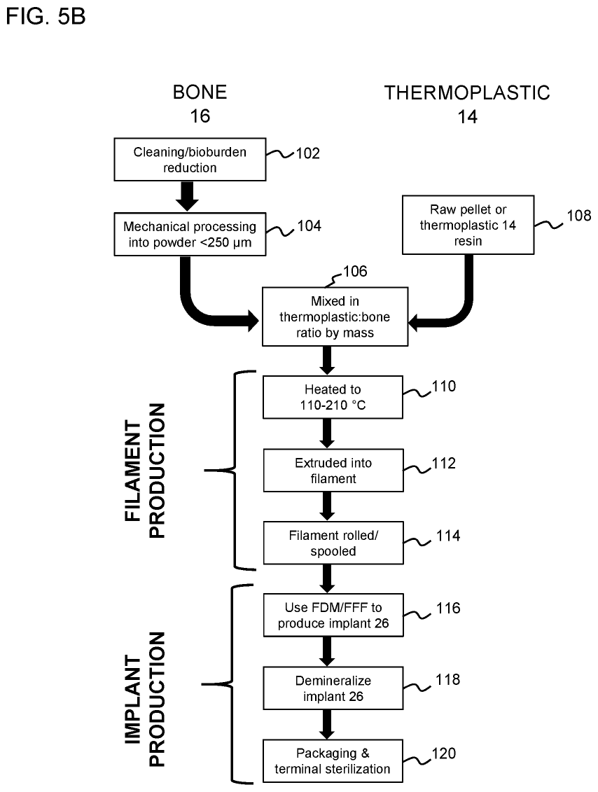

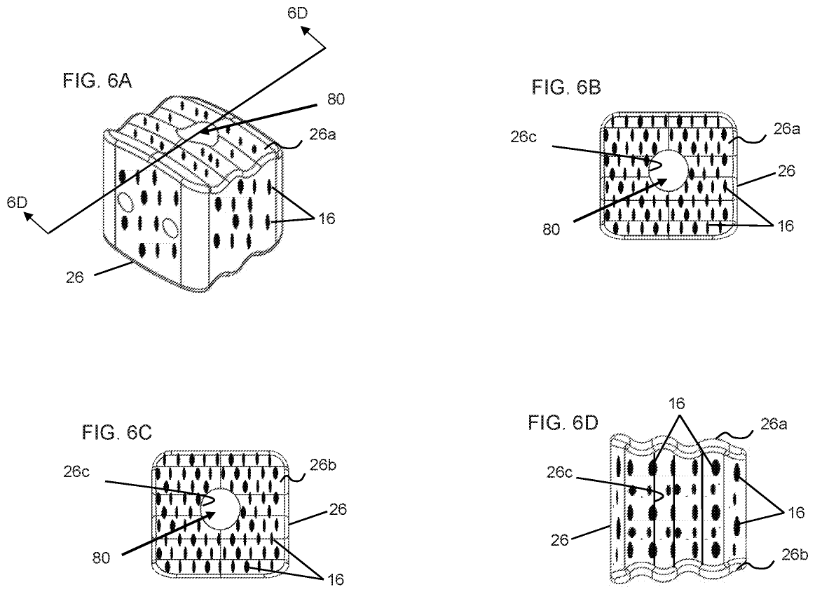

Referring now to FIGS. 1-8D, a system 10, device, implant and processes for generating or creating a bone-derived thermoplastic and bone mixture that is adapted and processed to create a filament that is subsequently used to produce a device, such as an implant, is shown and will now be described. For ease of understanding, the system 10 will be described relative to FIGS. 1-4, with associated processes being described relative to FIGS. 5A-5B. The implant is described in detail relative to the examples in FIGS. 6A-8D.

As best illustrated in FIG. 1, the system 10 comprises at least one or a plurality of stations for processing bone 16 and thermoplastic 14 together and, ultimately, to form a device/implant 26. A first station 12 includes a bone/thermoplastic processing and mixing station 12 for processing and/or mixing a thermoplastic 14 and bone 16 together. In general, the processing and mixing station 12 comprises multiple processes applied to the bone 16 to ensure proper ratio and distribution of the bone 16 with the thermoplastic 14. A cleaning/bioburden reduction process (station or block 12a), is performed to reduce bioburden in the bone 16. Thereafter, the bone 16 is mechanically processed into powder (station or block 12b) having particles having a predetermined or desired size. Preferably, the powder particles are less than about 1,000 μm, and in two other embodiments, the particles are 500 μm or smaller in size or 250 microns or less in size, respectively. These processes are described in more detail later herein relative to FIG. 3.

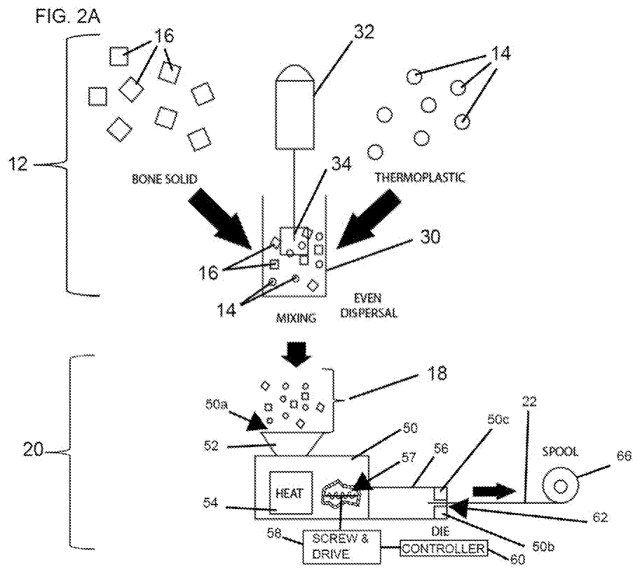

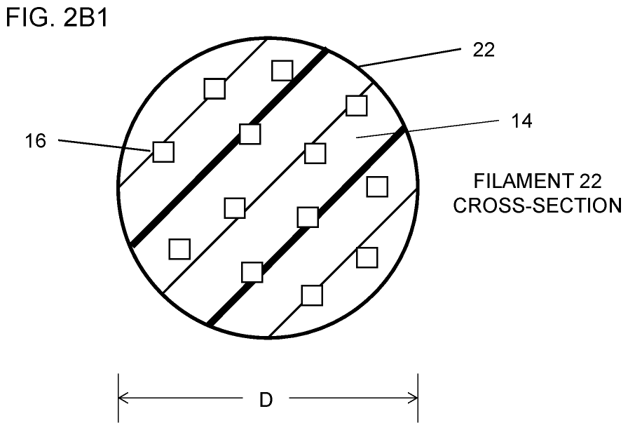

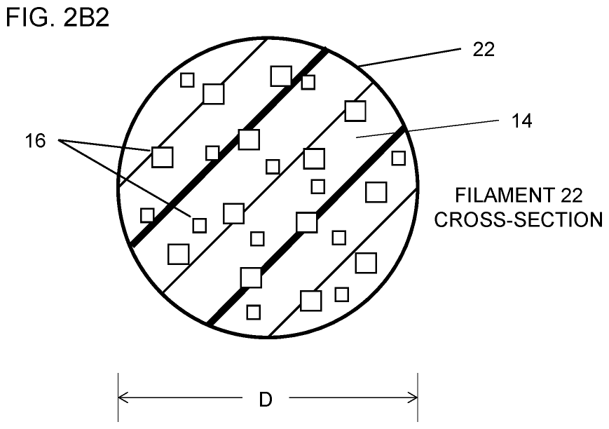

In general, at block 12c of station 12, the bone 16 and thermoplastic 14 are mixed in predetermined or desired ratios, which can and will vary depending upon the device/implant 26 characteristics and its application. The resultant bone/thermoplastic mixture 18 can then be used in subsequent processing or production steps. Further details of the bone/thermoplastic processing and mixing station 12 and related steps will be described later herein relative to FIGS. 2A and 3.

The bone/thermoplastic mixture 18 is supplied to a filament production station 20 (FIG. 1) wherein the bone/thermoplastic mixture 18 is heated to or above the thermoplastic's melting temperature (station or block 20a), extruded into a filament 22 (block 20b) and then collected onto a spool 66 (FIG. 2A). For example, the melting temperature for PMMA is on the or of about 160 degrees or higher. Thereafter, the finished bone/thermoplastic filament 22 is ready for further processing or use.

As best illustrated in FIG. 1, note that the bone/thermoplastic filament 22 is supplied to an implant/device production station 24 wherein a device, such as a finished surgical implant or other device 26, is produced. In general, the implant/device production station 24 may comprise a volumetric/3D fusion deposition modeling (FDM)/fused filament fabrication (FFF) printing system 70 (block 24a) described in more detail later herein.

The system 10 may further comprise one or more optional processing stations 28 for processing the finished surgical implant 26. Such operations may comprise bone exposing (e.g., sanding, solvents or the like), machining, sterilization or other processes. The finished surgical implant 26 is subsequently used for its intended purpose and in recognition of the characteristics of the finished surgical implant 26. For example, as will be described in more detail later herein, the finished surgical implant 26 may comprise bone components selectively positioned at predetermined areas anywhere in the device/implant 26. For example, note the bone 16 particles are located near an external surface 26a (FIG. 6A) of the finished surgical implant 26 in order to facilitate increasing osteoconduction when the finished surgical implant 26 is implanted. Accordingly, for this type of finished surgical implant 26, a user, such as a surgeon, orients such bony areas in the device/implant 26 in operative relationship with a patient's bone so that the bony component of the finished surgical implant 26 is in contact with the patient's bone, thereby facilitating increasing and improving osteoconduction. These and other features will be described in more detail later herein.

Referring now to FIGS. 1, 2A, 3, 4A and 4B, further details of the system 10 and processes will now be described. Prior to mixing, the bone 16 and a predetermined thermoplastic 14 are placed in a non-static mixing tank or container 30 (station 12 in FIG. 1, FIG. 2A) using a mixer 32 having an auger 34 as shown. In the illustration being described, the bone 16 and thermoplastic 14 are mixed in a dry form and agitated, stirred, shaken, ultrasonically vibrated or otherwise mixed such that the subsequent bone/thermoplastic mixture 18 comprises a substantially even dispersal of the bone 16 and the thermoplastic 14 to the predetermined or desired ratios. In general, the mixture is mechanically shaken until the bone is visibly dispersed in an apparent uniform manner, adhered on thermoplastic pellet surfaces by static. In the future this may be achieved in a more controlled method using a shaker or vortexer for a short timed duration. In several illustrative examples, PMMA was used as the thermoplastic 14 and mixed with bone 16 in ratios of 50:1, 16.67:1 and 10:1 were mixed using this method. The mixture was further processed as defined herein.

FIG. 3 illustrates further details, broken down into steps or substations, relating to the mixing processes that occur in one embodiment at the bone/thermoplastic processing and mixing station 12. As illustrated, the processing and mixing that occur at the mixing station 12 has a first step or sub-station 40 where the supply of bone 16 and thermoplastic 14 are provided for mixing. In the illustration being described, the bone 16 is cortical bone derived from a human (allograft) or animal (xenograft) source. Moreover, in the illustration, the thermoplastic 14 may be a resin or pellet and may comprise nylon, acrylonitrile butadiene styrene (ABS), polycarbonate, polyetherimide, polymethylmethacrylate (PMMA), acrylic, polyacryletherketones or the like. It should be understood that other types of polymers or thermoplastic resins may be used.

At substation or step 42 (FIG. 3), which was briefly referenced relative to block 12a in FIG. 1, the bone 16 and/or thermoplastic 14 may be further processed to remove bioburden in the form of blood and lipids and the like prior to mixing. The bone 16 is aseptically processed using thermal, mechanical, and/or chemical methods to remove blood and lipids and to reduce bioburden. Thermal processes may include exposure to heated solutions or other means to reduce bioburden. In one illustrative embodiment, a thermal process may be used to aseptically process the bone 16 by exposing the bone 16 to solutions that are heated between approximately 22° C.-50° C. Of course, other temperature ranges may be used, depending on the bioburden to be removed. One or more of these processes may be used to facilitate reducing bioburden. Mechanical processes may include ultrasonic, stirring, and/or shaking. Chemical processes may include physiologic saline, sterile water, detergents, isopropyl alcohol, hydrogen peroxide or antibiotics. Referring now to the substation or step 44 (FIG. 3), the bone 16 and/or thermoplastic 14 are mechanically processed into a powder or granulate. The step or substation 44 occurs at station 12b in FIG. 1. In this regard, in a preferred embodiment, the mechanical processing includes the process of grinding and sieving to a desired particle size.

The bone 16 is processed to get the bone 16 to the predetermined or desired particle size, which is dependent in part upon how the bone 16 is subsequently processed. For example, powder particles of 1,000 μm diameter or size or less are desirable for use in manufacturing methods such as injection molding. In another example, powder particles of 500 μm or less in size are desirable for use in manufacturing methods such as extrusion and volumetric printing. In yet another example, the particle size is less than 250 microns. In one illustrative embodiment, the particles in the range of 125-250 microns were used successfully. Different sizes may be suited for different application/implant 26 types, but again, in a preferred embodiment the size is desired to be less than or equal to 1,000 microns. Mammalian bone tissue is known to contain one or more proteinaceous materials, presumably active during growth and natural bone healing that can induce a developmental cascade of cellular events resulting in endochondral bone formation. The active factors are variously been referred to in the literature as bone morphogenetic or morphogenic proteins (BMPs), bone inductive proteins, bone growth or growth factors, osteogenic proteins, or osteoinductive proteins. These active factors are collectively referred to herein as osteoinductive factors.

At substation or step 46, the bone 16 material or particles may be further processed to reduce moisture to approximately less than or equal to 6% by weight. In one embodiment, moisture is removed by lyophilization. After the moisture level in the bone 16 material is at the desired or predetermined level, the bone 16 and/or thermoplastic 14 are now adapted and ready to be mixed to the desired ratio using the mixer 32 and auger 34 (FIG. 2A) mentioned previously. In this regard, the predetermined or desired ratio may depend upon the amount of bone 16 desired in the ultimate finished surgical implant 26. In general, the bone 16 and the thermoplastic 14 are weighed and then combined in a specific or predetermined ratio by mass, as shown in substation or step 48 (FIG. 3) which was generally described relative to block or station 12c in FIG. 1. In one illustrative embodiment, the mass of thermoplastic 14 is approximately two times the mass of the bone 16. It has been found that this results in the finished surgical implant or device 26 having a relatively high proportion of bone 16 material. This may be particularly useful when the implant or device 26 is desired to have a high level of bone 16 for improving osteoconductivity. The bone is mixed with the thermoplastic resin in a specific predetermined ratio. The ratio is determined by mass where the mass of thermoplastic resin 14 ranges from 2 to 100 times the mass of the bone 16. In another embodiment, the mass of the thermoplastic 14 is approximately ten (10) times the mass of the bone 16. In still another embodiment, the mass of the thermoplastic 14 is approximately fifty (50) times the mass of the bone 16. Thus, in a preferred embodiment, the mass of the thermoplastic in the predetermined ratio is approximately 10-50 the mass of the bone 16. It has been found that in applications where a relatively small amount of bone 16 is desired or is necessary, a higher percentage of thermoplastic 14 may be used. Thus, it should be understood that different ratios may be used. Lower concentrations are used in areas that require higher mechanical integrity that is similar to the polymer alone. Higher concentrations are used in non- or low-load bearing areas where bone growth is desired.

The substation or step 48 in FIG. 3 occurs at block or station 12c in FIG. 1, and after the substation or step 48 (FIG. 3), it should be understood that the bone/thermoplastic mixture 18, which has the bone 16 and thermoplastic 14 mixed and combined (block 50 in FIG. 3) in the desired or predetermined ratio that is desired for the application, are placed in the container 30 (FIG. 1) and then mechanically agitated and mixed by the auger 34 (FIG. 2A) at ambient or room temperature to thoroughly mix the two components. In the illustration being described, the container 30 is an antistatic container that facilitates such mixing. After mixing, the final bone/thermoplastic mixture 18 (FIGS. 1 and 3) is provided to stations 20 and 24 (FIG. 1) for further processing. At this point, the bone 16 is substantially evenly dispersed with the thermoplastic 14. As mentioned earlier, in a preferred embodiment, the mechanical processing includes mixing in a vortexer or a rotating drum designed to thoroughly and uniformly mix the components.

After the bone/thermoplastic mixture 18 is prepared, the mixture 18 is transferred to the filament production station 20 (FIG. 1) for further processing as will now be described. Referring back to FIGS. 1 and 2A, the system 10 comprises an extruder 50 (FIG. 2A) having an inlet 50a, a storage hopper 52, a conventional heater 54 and a barrel 56. The barrel 56 has a screw and drive 58 that is under the control of a controller 60.

As is conventionally known, the extruder 50 comprises an extrusion chamber 57 which receives the bone/thermoplastic mixture 18 from the hopper 52 after the mixture 18 is placed therein and a heater 54 heats the bone/thermoplastic mixture 18 up as it is being turned over by an internal screw and drive 58 and then fed into and through the barrel 56 where it exits an exit end 50b. Note that the extruder 50 comprises a die 50c having a die wall 50d that defines an extrusion orifice 62. In the illustration being described, it is important to understand that the orifice 62 may have a dimension, such as a diameter, that is of a predetermined size. This is why it is important that when the bone 16 is processed at the bone/thermoplastic processing and mixing station 12, the size of the bone 16 particles must be smaller than the extrusion orifice 62 in order to avoid possible clogging of the die 50c and to facilitate insuring that a consistent filament 22 is created. In the example, the extrusion orifice 62 has a diameter of less than about 3 mm and the bone 16 particles are preferably less than about 1,000 microns in diameter.

After the bone/thermoplastic mixture 18 is loaded into the hopper 52, which feeds the mixture 18 into the extrusion chamber 57 (FIG. 1), the controller 60 of the extruder 50 energizes the heater 54 to create heat to heat the mixture 18 to a predetermined temperature that is adequate to melt the bone/thermoplastic mixture 18 into a flowable state so that it ultimately can be extruded through the extrusion orifice 62 of the die 50c to create the filament 22. This is what occurs at station 20a (FIG. 1) and 20b in station 20 described generally earlier herein. In one illustrative embodiment, the heater 54 heats the chamber 57 and barrel 56 to a predetermined temperature of 160° C. or greater, and the temperature may depend in part of the melting point of the thermoplastic 14 used.

As illustrated in FIG. 1, the filament 22 is preferably a continuous filament that is collected and wound (substation 20c in FIG. 1) on a supply spool 66 for later use at the implant/device production station 24 while implant 26 production and processing occurs.

As mentioned earlier, the bone/thermoplastic mixture 18 is heated as it passes and is driven through the barrel 56 by the internal screw and drive 58 and heated for a predetermined period of time. As the bone/thermoplastic mixture 18 passes through the extruder 50, the bone/thermoplastic mixture 18 liquefies and is mixed by the internal screw and drive 58 which is rotatably driven at a predetermined speed that is appropriate for making the extruded filament 22 of a desired diameter D (FIG. 2B1). Note that the bone 16 material is preferably substantially evenly distributed through the filament 22 in the ratio chosen in the illustration in FIG. 2B1.

FIG. 2B2 illustrates another embodiment of the filament, which as mentioned herein may have different quantities and ratios of thermoplastic 14 to bone 16 material, and may even have different types and sizes of both thermoplastic 14 and bone 16. For example, note in FIG. 2B2, a filament 22 is shown having bone 16 particles of different sizes and shapes.

The final finished filament 22 is shown in cross-section in FIG. 2B1 and is comparable in shape and flexibility to a conventional sport fishing line with bone 16 particles suspended in the thermoplastic 14 and is gathered on the spool 66 as mentioned earlier. Note the substantially even distribution of bone 16 solid material captured and distributed with the thermoplastic 14 in the filament 22. In the illustration being described, the filament 22 comprises the diameter D (FIG. 2B1). The filament 22 can be terminally sterilized using conventional sterilization means for biologic and medical devices. Such sterilization may be in the form of irradiation, heat or chemical sterilization in a manner that is conventionally known.

As mentioned earlier, the filament 22 is collected on the spool 66 (substation 20c in FIG. 1) and is ready for use at the implant/device production station 24 mentioned earlier whereupon the filament 22 may be used to produce the implant or device 26.

Advantageously, the extruder 50 heats and liquefies the thermoplastic 14 as the internal screw and drive 58 mixes the liquefied material with the bone 16 at the predetermined speed appropriate for making and extruded filament 22 of a desired diameter. As mentioned, FIG. 2B1 illustrates this diameter D, and in one preferred embodiment, the preferred filament 22 diameter D is approximately about 2.5 mm to 2.9 mm. In another illustrative embodiment, the diameter D is approximately about 1.6 mm to 1.8 mm. In general, the filament 22 size selected will depend upon the subsequent processing steps and the desired characteristics of the device/implant 26.

The following are several examples of the production of the filament 22 using the extruder 50. In one example, the bone/thermoplastic mixture 18 having powder particles of 125-250 microns evenly dispersed with thermoplastic 14 and fed into the bin or hopper 52 of the extruder 50. The heater 54 heated the material to 190° C. in one illustration.

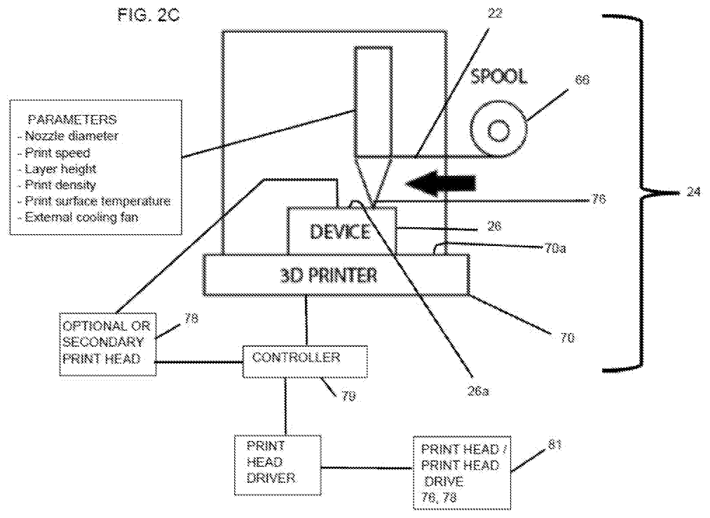

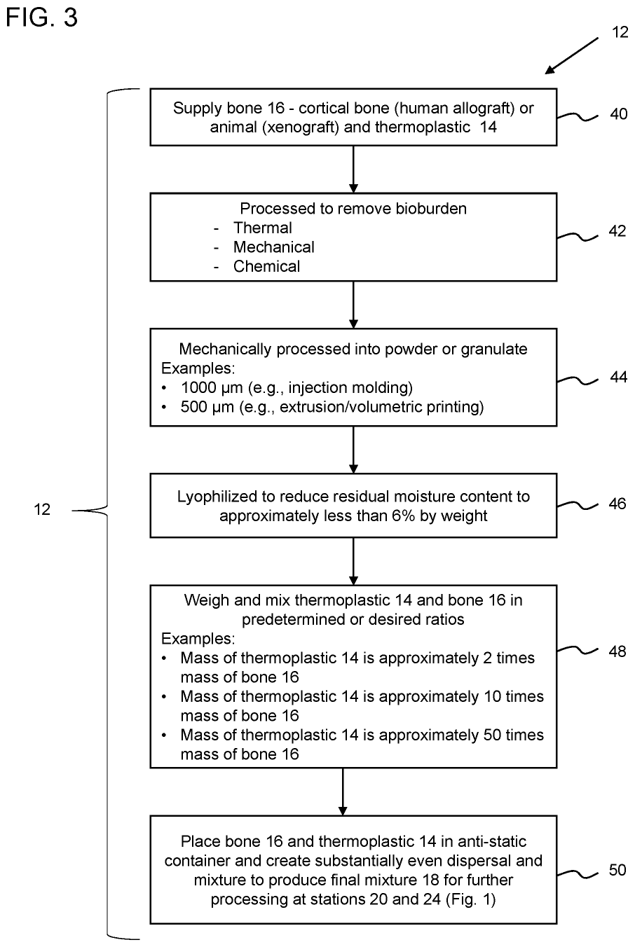

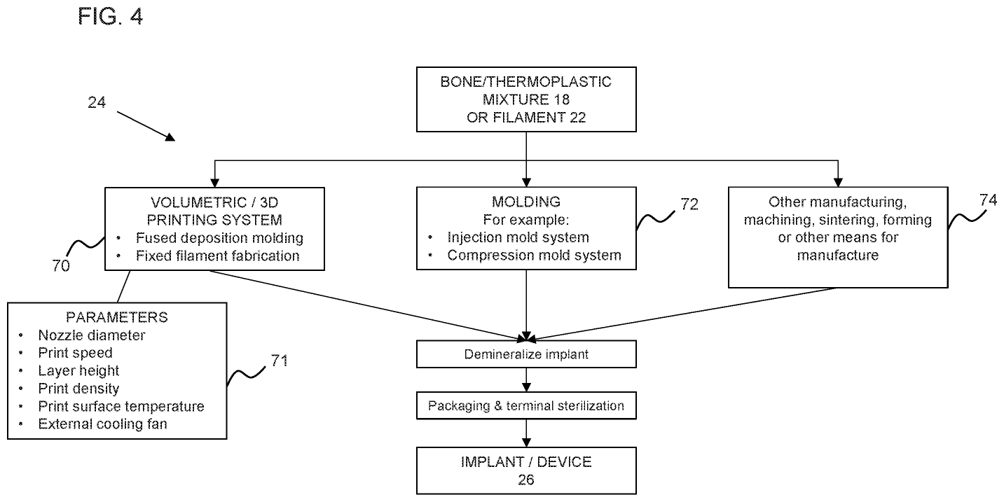

As mentioned earlier, the filament 22 is collected on the spool 66 and the inventory of filament 22 is provided to the implant/device production station 24 (FIG. 1) where it can be further processed. FIG. 1 illustrates several general systems or steps (block 24a, 24b and 24c) in a preferred embodiment, and FIGS. 4 and 5A-5B are views showing other illustrative methods or processes for forming or manufacturing the implant/device 26. In this regard, the implant/device production station 24 comprises at least one or a plurality of systems or processes for manufacturing the implant/device 26. Referring to FIG. 1, station 24 and FIG. 4, the implant/device production station 24 receives at least one of the bone/thermoplastic mixture 18 or the filament 22 for subsequent processing. As shown and described earlier, the station 24 may comprise the volumetric/3D printing system 70, such as fused deposition molding (FDM) device or a fused filament fabrication (FFF) device, commonly referred to as “3D printing”.

In addition or alternatively, the station 24 may also comprise other means for forming the implant/device 26, such as an injection mold system. The station 24 may also comprise other forms of machinery, devices, nozzles or print heads for forming the implant/device 26 and which may include sintering, machining or the like. It is important to note that in a preferred embodiment, the filament 22 is used for processing and manufacture of the implant/device 26, but there may be applications where the bone/thermoplastic mixture 18 from the bone/thermoplastic processing and mixing station 12 may be used directly in one or more of the manufacturing processes. In a preferred embodiment, at least one of the fused deposition modeling (FDM) or fused filament fabrication (FFF) methods is used, and this will now be described relative to FIGS. 2C and 4.

In a first example, the filament 22 having bone 16 and PMMA as the thermoplastic 14 is loaded into the printing system 70 (FIG. 2C). In the illustration being described, the volumetric/3D printing system 70 heats the filament 22 above its melting point to create a flowable mixture. In the illustration, the temperature is between approximately greater than 160° C., but it should be appreciated that lower or higher temperatures may be utilized depending on the characteristics of the filament 22, and particularly, the thermoplastic 14 in the filament 22. Continuing with the example, the volumetric/3D printing system 70 applies the heat for a relatively short duration of time. It is important to note that it is desired to minimize the thermal exposure to the bone 16 portion of the filament 22, which can have undesirable impact on the bone 16 characteristics when it appears in the final implant 26.

Referring back to FIG. 2C, note that the volumetric/3D printing system 70 comprises a nozzle or print head 76 that receives the filament 22 and heats it to a flowable state under the control of a conventional controller and driver (not shown). For the fixed filament 22 fabrication, the implant/device 26 is produced conventionally by driving the print head 76 to print consecutive layers of liquefied mixture 18 material, which subsequently solidifies upon cooling to form the implant/device 26.

As is conventionally known, the print head 76 is driven at a predetermined speed across a print surface 70a (FIG. 2A) which is a surface, such as a bone-engaging surface 26a (FIG. 6A). As is conventionally known, a controller 79 is adapted to control the nozzle or print head 76 which is situated a predetermined distance from the print surface 70a and driven such that a print layer (not shown) is formed. Multiple print layers are laid in order to form the implant/device 26. As shown in FIG. 4, the volumetric printing system 70 comprises at least one or a plurality of parameter controls for controlling various parameters 71 in the volumetric/3D printing system 70. In this embodiment, these parameters comprise nozzle diameter, print speed, layer height, print density, print surface temperature, cooling and the like.

In one illustrative embodiment, the nozzle diameter is 0.8 mm and the print speed was 15-30 mm/second. A print layer height, which corresponds to the height or print layer thickness between the print surface 70a (FIG. 2C) and the top surface 26a of a first printed layer, is on the order of about 0.2-0.4 mm. The print density is approximately 100%. In general, the finished printed implant/device 26 are defined by an outer shell and then the interior section. The user controls the wall thickness. The shell is printed at maximum (100%) density, so that there are no gaps/voids/pores (unless included as design elements). The remaining interior can then be printed at a user defined density. For example, an implant 26 may be printed at 20% for speed and cost, meaning 80% of the object's interior is substantially empty or void. In the illustration above however, the implant 26 was printed at 100% because of the desire to have a solid, mechanically robust implant. In the illustration being described, the print surface 70a (FIG. 2C) was initially heated to a temperature of 100° C. and then gradually reduced to 40° C. which facilitates hardening the print layers when they are applied. The print speed was approximately 15-30 mm/second and the nozzle diameter was 0.8 mm in this example. An optional cooling fan (not shown) may be used to facilitate cooling the implant 26 during manufacture, but in the illustration being described, it was not.

After the implant 26 is completely manufactured and removed from the volumetric/3D printing system 70, the system 10 may have further implant processing steps as referred to earlier relative to the block or station 28 in FIG. 1, such as machining, bone-exposing (e.g., via sanding or etching) deburring, etching, sanitizing or the like.

It is important to note that during implant/device 26 production, the bone 16 carrying filament 22 liquefies and the bone 16 particles flow with the thermoplastic 14 through the nozzle or print head 72. As a result of the substantially even dispersal of bone 16 in the filament 22, substantially even dispersal and distribution of bone 16 particles in the finished implant 26 is provided. It is important to note that the volumetric/3D printing system 70 may comprise one or more optional or additional nozzles or print heads 78 so that multiple materials or filaments 22 can be simultaneously used to print or manufacture the implant/device 26. For example, filaments 22 having different characteristics, such as different bone 16 to thermoplastic 14 resin ratios, may be used. In one example, a first filament 22 may contain a specific ratio of bone 16 to thermoplastic 14 of about 50:1, while another filament 22 used in the optional nozzle or print head 74 may have a bone 16 to thermoplastic 14 ratio of 10:1. It is important to note that the resultant implant 26 can be manufactured and customized to have unique predetermined characteristics because of the ability to control and utilize multiple filaments 22 having different characteristics. In one illustrative embodiment, the filament 22 having a relatively large ratio of bone 16 to thermoplastic 14 resin will cause more bone 16 particles to be layered onto the implant 26, whereas a filament 22 that has a comparatively less bone 16 to thermoplastic 14 ratio will impart less bone 16 onto or into the implant 26 during production. Because the nozzles or print heads 74 are under the control of a controller (not shown), they can also be controlled to control the dispersion of the bone 16 in the finished implant 26.

In another illustrative embodiment the system 10 comprises at least one or a plurality of other print heads 78 that are also under the control of controller 79 and print head drive 81. Each print head 76, 78 utilizes filaments 22 having different characteristics loaded into the nozzles or print heads 76 and 78. A first filament 22 comprises a specific ratio of bone 16 to thermoplastic 14 and a second filament 22 is a support material or simply pure thermoplastic 14 without any bone 16 at all. The controller 79 selectively drives and controls the nozzles or print heads 76 and 78. The areas in the implant 26 where the filament 22 with bone 16 is printed will deliver bone 16 to the implant 26 in the specific areas where the nozzles or print heads 76 and 78 lay the heated thermoplastic 14 and bone 16 molten suspension.

In another embodiment, the filaments 22 used during printing could each have the same bone 16 to thermoplastic 14 ratio or the two filaments 22 may contain bone 16, but at different ratios. Alternatively and as mentioned, one filament 22 may contain no bone 16 and only thermoplastic 14 while another filament 22 has a ratio of bone 16 and thermoplastic 14. Although not shown, the system 10 may comprise a supply of filaments 22 having different characteristics for ease of use by a user.

For ease of understanding and to emphasize the various process steps mentioned, FIGS. 5A and 5B are two illustrative summaries of the processes previously described herein relative to FIGS. 1-4. In FIG. 5A, bone 16 is provided and at block 82 heat, acid or mechanical selective removal of protein components is performed in this embodiment. It should be understood that in subsequent embodiments and as described earlier herein, the demineralization may occur during the implant production stage, rather than in the bone preparation stage. In a preferred embodiment, the demineralization occurs at the implant production stage, not the bone preparation stage. At block 84, the bone 16 is mechanically processed into a powder, granule/fiber and then it is mixed with raw pellet or thermoplastic 14 resin as shown at block 88. Note that the mixing could be performed either hot or cold. After the bone 16 and thermoplastic 14 are mixed, they are subjected to heating and pressurization at block 90 and then submitted to the extrusion process described earlier at block 92. The filament 22 rolling is performed at the filament production station 20 at block 94 and, if necessary, packaging and terminal sterilization is performed on the filament 22 at block 96. Thereafter, the filament 22 is used in the 3D mechanical device (block 98) or other manufacturing method as mentioned earlier and then the implant 26 is produced (block 100).

Another illustrative process is shown and described relative to FIG. 5B. In this illustrative embodiment, the bone 16 is cleaned and bioburden reduction is performed at block 102. Mechanical processing into powder of less than about 250 micrometers at block 104 is performed and then the bone 16 is mixed with the thermoplastic 14 at block 106. The bone/thermoplastic mixture 18 is heated at block 110 to between approximately 110° C.-210° C. as described earlier herein and extruded into the filament 22 (block 112) and then rolled and spooled (block 114). It should be understood that the blocks 102-106 comprise the bone and thermoplastic mixing station or stage that occurs at station 12 (FIG. 1) while blocks 110-114 illustrate the filament 22 production stage that occurs at station 20 in FIG. 1. The process continues to block 116 where fused deposition molding (FDM) or fixed filament fabrication (FFF) are used to produce the implant 26 as described earlier relative to station 24.

Importantly, at block 118, the implant 26 is demineralized as described earlier. Note that this demineralization of the implant 26 occurs after thermal exposure during the manufacturing process and the filament 22 production process. This facilitates the bone mineral component to provide thermal protection to the osteoinductive bone proteins, thereby avoiding protein denaturation during heating. Thereafter, the implant 26 is packaged and sterilized and the like at block 120 and then distributed for use.

Advantageously, utilizing filaments 22 with the same bone 16 to thermoplastic 14 ratios or utilizing different filaments 22 having different materials or ratios of materials of if both have bone 16 facilitates customizing and manufacturing the implants 26 to have predetermined bone patterns which will now be described relative to FIGS. 6A-8D.

FIGS. 6A-6D show a first illustrative implant 26 made according to the system 10 and processes set forth herein. It is important to note that the implant 26 is a bio-active, osteoconductive surgical implant that supports bone growth. These attributes are imparted into the implant 26 by the incorporation of the human or animal-derived bone 16 element. In the first illustrative embodiment of FIGS. 6A-6D, note that the bone 16 is distributed uniformly throughout the implant 26 as shown.

Most importantly and as alluded to earlier herein, some embodiments include implants 26 where the bone 16 is strategically located in areas where bioactivity is desired. For example, in the implant 26 illustrated in FIGS. 6A-6D, the implant 26 is a spinal cage that is adapted for placing between adjacent vertebra (not shown) of a patient. Preferably, after the cage is situated between the vertebra, the bone-engaging surfaces 26a and 26b engage directly with the adjacent vertebra, respectively, of the patient. Alternately, the device or implant 26 could be another type of implant, such as a rod, screw, prosthetic, implant, or the like. In this application, it is important that the implant 26 have bone 16 areas strategically located where the bioactivity is desired, which is the junction area or surfaces 26a and 26b where the implant 26 engages the patient's bone.

Advantageously, the invention contemplates selective bone 16 placement in the ultimate device/implant 26. For example, for a device 26 intended as use for a spinal fusion implant, bone 16 is concentrated on the devices superior and inferior surfaces 26a and 26b to interact with adjacent vertebral bodies. This further facilitates osteoconductivity between the surgical implant 26 and the vertebral bodies as mentioned. The localized areas of bone 16 further facilitate or aid in directing a desired biological response.

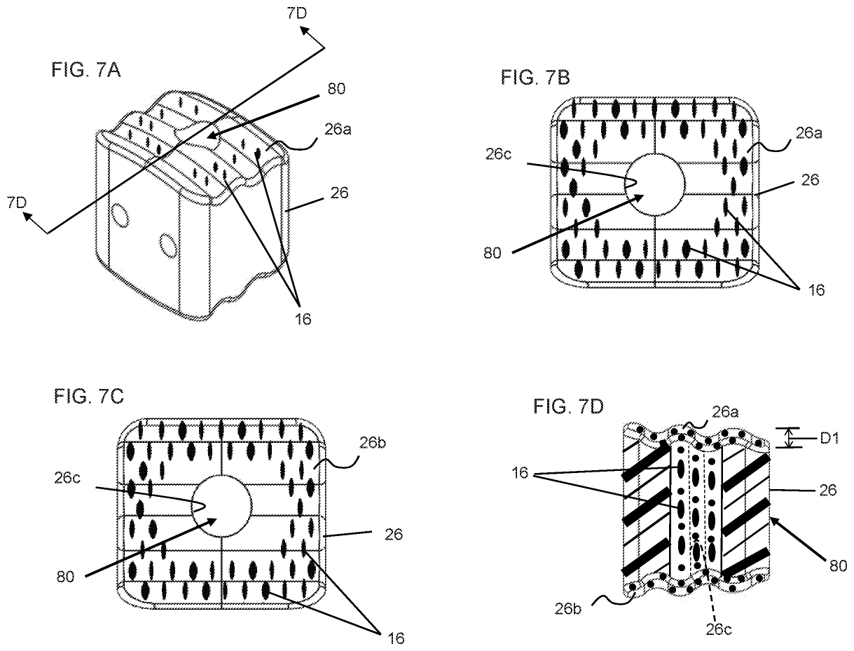

FIGS. 7A-7D provide several illustrative embodiments where the bone 16 is exposed on the surfaces 26a and 26b of the implant 26 to enhance osteoconductive and/or osteoinductive properties. In the illustration being described, the bone 16 particles may be exposed on the surfaces 26a (FIG. 7A) and 26b of the implant 26. If desired, the implant 26 may be further processed (as mentioned earlier relative to station or block 28 in FIG. 1) to expose more bone 16. In this regard, bone 16 can be exposed by mechanical methods, such as an abrasive sanding or chemically, such as by etching. For example, bone 16 can be exposed by contacting the implant 26 with solvents or solutions to remove a portion of the thermoplastic 14 while retaining the bone 16 on the surface 26a of the device 26. In still another example, the implant 26 can be exposed to acids known to demineralize bone, thereby making osteoinductive proteins, such as bone morphogen protein (BMP) available. These alternatives are shown at block 28 in FIG. 1.

In one embodiment, it may be desired to demineralize the device or implant 26 prior to use. In one example, the implant 26 is soaked in 0.5 N hydrochloric acid (HCl) for two subsequent 45 minute cycles with mechanical agitation on a stir plate at ambient temperature to facilitate exposing bone 16. The implant 26 is rinsed thoroughly with sterile water or saline and neutralized to physiologic pH with sterile buffered saline. It is important to note that in this example, the demineralization occurs after thermal exposure during the manufacturing processes at the filament production station 20 and during the implant device production station 24 in order for the bone mineral components of the bone 16 to provide thermal protection to the osteoinductive bone proteins, thereby avoiding protein denaturation during heating. It is believed that this sequence is important because it avoids undesired protein denaturation resulting from heating bone. If such process occurred prior to or during production of the implant 26, the heating may cause an undesirable denaturation of the bone protein.

The implant 26 possesses hygroscopic properties due to the inclusion of bone 16, which facilitates absorbing fluid from its surroundings. In the illustration being described, the absorbed fluid may contain nutrients and/or cells that further facilitate a healing response after the implant 26 is implanted in a patient, for example. Thus, the implant 26 comprises biomechanical properties appropriate for its intended use and can accommodate relevant physiological loading without failure. For example, the implant 26 is further processed at (block 28 in FIG. 1) utilizing strain hardening, compression annealing, cross-linking, addition of strengthening additive, or similar means to bolster or alter the device/implant 26 biomechanical properties. It is important to note that the bone 16 content influences biomaterial properties in a very controlled and predetermined manner. For example and as previously mentioned, the implant 26 may possess regions of lower bone 16 content in the implant 26 to emphasize a mechanical attributes of the thermoplastic 14. Alternatively, in another example, the implant 26 may possess regions of higher bone 16 content to impart more bone-like mechanical qualities.

As mentioned earlier, FIGS. 6A-6D shows an implant 26 modified or produced with bone 16 evenly dispersed in the thermoplastic 14 and therefore evenly dispersed throughout the implant 26. It should be understood that in view of the control of the manufacturing process and the illustration the 3D printing process, it is also contemplated that bone 16 may be situated only a certain depth D1 (FIG. 7D) of the top surface 26a, such as 1-4 mm selective application using a filament 22 having bone 16. In one embodiment, this depth D1 may be located specifically in respect to the portion of the implant 26 tat is directly opposing the device-patient interface. For example, note a middle portion or area 80 of the implant 26 in FIG. 7D, may be printed with the optional or secondary nozzle or print head 74 using a filament 22 that has only thermoplastic 14 or other material, but no bone 16. FIGS. 7A-7D illustrate the implant 26 with bone 16 components selectively located near the external surfaces 26a and 26b which facilitates increased osteoconduction when the implant 26 is implanted into a patient.

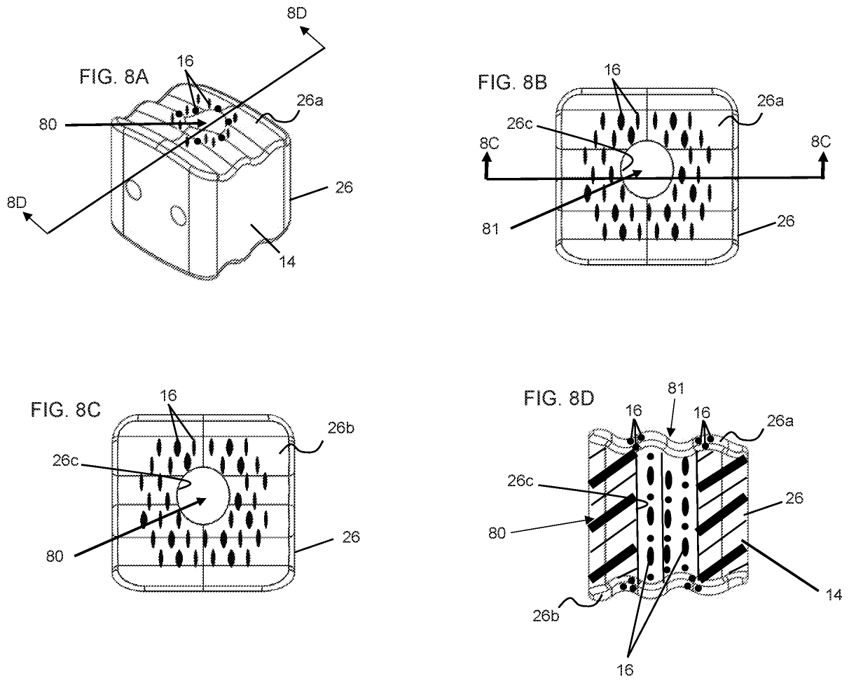

FIGS. 8A-8D show another illustrative implant 26 with the bone 16 components selectively positioned and located near an internal surface, such as internal wall surface 26c, for increased mechanical strength and osteoconductive properties. In this regard, the internal surface 26c may define an aperture 81 (FIG. 8D). In this illustration, the bone 16 component is selectively located near the internal wall surface 26c and surface 26a, 26b for increased mechanical strength or other biomechanical characteristics.

It should be appreciated that these examples are merely illustrative and, again, the biomechanical, osteoconductive and/or osteoinductive properties of the device/implant 26 may selectively change if desired and depending on the application.

### Other Considerations

1. It should be appreciated that air or other gas may be injected into the thermoplastic mixture 18 during a preparation, heating, mixing or extrusion process to create a porous structure upon cooling.

This invention, including all embodiments shown and described herein, could be used alone or together and/or in combination with one or more of the features covered by one or more of the claims set forth herein, including but not limited to one or more of the features or steps mentioned in the Summary of the Invention and the claims.

While the system, apparatus and processes herein described constitute preferred embodiments of this invention, it is to be understood that the invention is not limited to this precise system, apparatus and processes, and that changes may be made therein without departing from the scope of the invention which is defined in the appended claims.

What is claimed is:

1. A method of generating a bone-derived thermoplastic extrusion utilizing the mechanical combination of human or animal bone solid with at least one thermoplastic resin, such that there is uniform dispersal of the bone solid in the resin;
the extrusion process comprising material pressure and heating upon a die, mold or runner to create a net shape;
the extrusion comprising filament, pellet, bar, molding, three dimensional printing material stock, or similar structures;
the bone proteins compromising collagen, bone morphogenetic proteins, osteocalcin, sialoprotein, osteopontin, osteonectin and other structural and functional proteins of bone.

2. The method of claim 1, wherein bone is mixed with thermoplastic pellet in the solid state, undergoing mechanical agitation prior to or during the extrusion process;
the mixing with the thermoplastic below the glass transition temperature of the thermoplastic;
the mixing further comprising physical agitation, electrostatic adhesion, or ultrasonic means to create a uniform dispersal of bone and thermoplastic solids.

3. The method of claim 2, wherein bone is combined with thermoplastic solid and agitated within an extrusion chamber subjected to heat, and/or pressure by auger screw or similar means to create dispersal of the bone solid in the forming extrusion.

4. The method of claim 1, wherein bone is mixed with thermoplastic pellet in the liquid state, undergoing mechanical agitation prior to or during the extrusion process.

5. The method of claim 4, wherein bone solid is combined with heated thermoplastic liquid and mechanically mixing to create uniform dispersal prior to being placed in an extrusion chamber for extrusion process;
the mixing comprising impeller agitation, ultrasonic agitation or other mechanical means resulting in a heated liquid state, with temperatures above the melting point of the thermoplastic, where the bone is added during and/or prior to the agitation and/or heating.

6. The method of claim 1, wherein the bone comprises mineral bone solid derived from human or animal bone, the bone treated via thermal, mechanical, or chemical processes to remove blood and lipids to reduce bioburden, leaving solid mineral components.

7. The method of claim 6, wherein the mineral components provide thermal stabilization to bone proteins, allowing for the proteins to avoid denaturation during extrusion heating.

8. The method of claim 1, wherein the bone is mechanically processed to create powdered, granular, elongate, or fiber form, with powder or granular forms having particles less than 1,000 μm in size, residual moisture content less than 6% by weight.

9. The method of claim 1, wherein the bone is mixed with thermoplastic resin in a specific ratio, the ratio is determined by mass, where the mass of thermoplastic resin ranges from 2 to 100 times the mass of the bone.

10. The method of claim 1 wherein the heating is applied for a short duration of time as to minimize thermal exposure to the bone solid.

11. The method of claim 1, wherein air or other gas is injected into the thermoplastic mixture during a preparation, heating, mixing or extrusion process to create a porous structure upon cooling.

12. The method of claim 1 wherein the filament is substantially flexible, such that it can be rolled onto a spool for handling and storage.

13. The method of claim 1 wherein the extrusion undergoes terminal sterilization via irradiation, heat or chemical means.

14. A bone-derived thermoplastic extrusion comprising a solid derived from human or animal bone;
the bone combined with a thermoplastic resin such that there is uniform dispersal of the bone solid in the resin;
the extrusion comprising filament, pellet, bar, molding, three dimensional printing material, or similar structures.

15. The extrusion of claim 14 wherein the bone comprises cortical bone powder, granule or fiber and is treated via thermal, mechanical, or chemical processes to remove blood and lipids and reduce bioburden, leaving solid mineral components.

16. The extrusion of claim 14 wherein the thermoplastic resin comprising nylon, acrylonitrile butadiene styrene (ABS), polycarbonate, polyetherimide, polymethylmethacrylate (PMMA), acrylic, polyacryletherketones or similar biocompatible thermoplastic.

17. The extrusion of claim 14 wherein the extrusion contains a minimum of 1% bone solid by weight.

18. The extrusion of claim 14 wherein the extrusion comprises a filament being substantially flexible, such that it can be rolled onto a spool for handling and/or optimized for use with volumetric manufacturing methods.

19. The extrusion of claim 14 wherein the extrusion undergoes terminal sterilization via irradiation, heat or chemical means.

20. An osteoconductive surgical implant manufactured from a bone-derived thermoplastic extrusion;
the surgical implant incorporating a combination of human or animal bone-derived solid and thermoplastic with dispersal of the bone in the thermoplastic.

21. The surgical implant of claim 20 manufactured utilizing volumetric printing, injection molding, machining, sintering, forming or similar means.

22. The surgical implant of claim 20 wherein there is substantially uniform dispersal of the bone component within the thermoplastic component.

23. The surgical implant of claim 20 wherein at least a portion of the bone-derived solid is exposed at the surface of the implant;
the exposed bone-derived solid expressing osteoconductive and/or osteoinductive properties and imparting the properties to the implant.

24. The surgical implant of claim 23 wherein the bone-derived solid on specific surfaces exposed in a controlled manner by mechanical or chemical means for exposure of osteoconductive or osteoinductive elements where biologic response is desired;
the chemical means comprising treatment of bone with acid such as acetic acid, citric acid, ethylenediamine tetraacetic acid, or hydrochloric acid.

25. The surgical implant of claim 20 wherein the implant comprises hygroscopic properties allowing for cellular and/or chemical diffusion and/or communication between internal bone-derived solids and the external implant surface.

26. The surgical implant of claim 20 wherein the implant is process-strengthened utilizing strain hardening, compression annealing, cross-linking, addition of strengthening additive, or similar means in order to accommodate physiological loading without failure.

27. The surgical implant of claim 20 wherein the implant possesses variable zones of differing bone content to impart regional mechanical and biological functions such as a diffusion gradient for directed biologic response.

28. A bone-derived thermoplastic filament comprising:
a human bone allograft, the bone allograft comprising mineral component and heat-resistant protein component, combined with a thermoplastic resin such that there is even dispersal of the bone allograft in the resin, heated and extruded to filament or pellet form;
the bone allograft comprising a proteinaceous component;
the proteinaceous component comprising mineralized collagen or other heat-resistant proteins;
the thermoplastic resin comprising nylon, nylon, acrylonitrile butadiene styrene (ABS), polycarbonate, polyetherimide, polymethylmethacrylate (PMMA), acrylic, polyacryletherketones or similar biocompatible thermoplastic;
the bone allograft comprising cortical bone powder, granule or fiber;
the mixture of thermoplastic and bone allograft being a molded from or extrusion into a filament or pellet;
the filament or pellet containing a minimum of 1% bone allograft by weight;
the bone allograft form having a diameter no greater than 70% of the filament or pellet diameter;
the filament being substantially flexible, such that it can be rolled onto a spool for shipping, handling and/or further manufacture;
the filament adapted for the manufacture of medical devices using volumetric manufacturing methods, such as three dimensional printing;
the filament, pellet and/or filament spool undergoing a terminal sterilization and packaging process via irradiation, heat or chemical means;
the filament, incorporated into a medical device using volumetric manufacturing process, such as three dimensional printing.

29. A bone-derived thermoplastic filament comprising:
a human bone allograft, the bone allograft comprising a mineral component combined with a thermoplastic resin such that there is even dispersal of the bone allograft in the resin, heated and extruded to filament or pellet form;
the thermoplastic resin comprising nylon, acrylonitrile butadiene styrene (ABS), polycarbonate, polyetherimide, polymethylmethacrylate (PMMA), acrylic, polyacryletherketones or similar biocompatible thermoplastic;
the bone allograft comprising cortical bone powder, granule or fiber;
the mixture of thermoplastic and bone allograft being a molded from or extrusion into a filament or pellet;
the filament or pellet containing a minimum of 1% bone allograft by weight;
the bone allograft form having a diameter no greater than 70% of the filament or pellet diameter;
the filament being substantially flexible, such that it can be rolled onto a spool for shipping, handling and/or further manufacture;
the filament adapted for the manufacture of medical devices using volumetric manufacturing methods, such as three dimensional printing;
the filament, pellet and/or filament spool undergoing a terminal sterilization and packaging process via irradiation, heat or chemical means;
the filament, incorporated into a medical device using volumetric manufacturing process, such as three dimensional printing.

30. A method of generating a thermoplastic filament or pellet by the following means:
the bone allograft mechanically processed to create powdered, granular, elongate, or fiber form;
mixing the bone allograft with a thermoplastic resin, in a liquid or allograft process, such that there is even dispersal of the bone allograft in the resin;
the mixing of the bone allograft with thermoplastic resin in proportions which maximize the proportion of bone by weight, while maintaining adequate mechanical properties of the resulting biomaterial;
the mixture of thermoplastic and bone allograft being heated to create a liquefied composite, the composite being pressurized and formed through a die, mold, or similar means to create the filament or pellet;
the mixing occurs in a heated state, with temperatures in excess of the melting point of the thermoplastic;
the mixing comprising impeller agitation or ultrasonic agitation or other means;
the mixing in a cool allograft state, where bone derived allograft is mixed with the thermoplastic below melting temperature of the thermoplastic;
the mixing in a solid state comprising thermoplastic granules and bone derived allograft granules of substantially similar size and surface volume;
the mixing in a solid state comprising physical agitation, ultrasonic means, to create an even dispersal of bone and thermoplastic allografts;
the method performed in a substantially sterile environment, such as a clean room;
the filament, pellet and/or filament spool undergoing a terminal sterilization and packaging process via irradiation, heat or chemical means;
the filament and/or pellet incorporated into a three dimensional manufacturing process.

31. A filament adapted for use in a volumetric or 3D printer or mold, said filament comprising:
a thermoplastic of a first predetermined quantity; and
processed bone of a second predetermined quantity;
said first and second predetermined quantities being selected to define a desired ratio of bone to thermoplastic in response to a desired amount of bone in an implant manufactured using the filament.

32. The filament as recited in claim 31, wherein said processed bone is at least one of sterilized or processed to reduce bioburden in said processed bone before it is added to said thermoplastic.

33. The filament as recited in claim 31, wherein said processed bone is distributed substantially evenly with said thermoplastic in predetermined areas of the filament.

34. The filament as recited in claim 31, wherein said processed bone is distributed substantially evenly with said thermoplastic substantially throughout the filament.

35. The filament as recited in claim 31, wherein said processed bone has a particle size of less than 1,000 μm.

36. The filament as recited in claim 31, wherein a mass of said thermoplastic is approximately two times a mass of said processed bone in said filament.

37. The filament as recited in claim 31, wherein said processed bone comprises mineral bone solid derived from human or animal bone, said processed bone treated via thermal, mechanical, or chemical processes to remove blood and lipids to reduce bioburden, leaving solid mineral components.

38. The filament as recited in claim 37, wherein said solid mineral components provide thermal stabilization to bone proteins, allowing for said bone proteins to avoid denaturation during heating.

39. The filament as recited in claim 31, wherein said processed bone is mechanically processed to create powdered, granular, elongate, or fiber form, with powder or granular forms having particles less than 1,000 μm in size, residual moisture content less than 6% by weight.

40. The filament as recited in claim 31, wherein said processed bone is mixed with said thermoplastic in a specific ratio, the ratio is determined by mass, where the mass of said thermoplastic ranges from 2 to 100 times the mass of said processed bone.

41. The filament as recited in claim 31, wherein said processed bone is mixed with said thermoplastic in a specific ratio, the ratio is determined by mass, where the mass of said thermoplastic ranges from 10 to 50 times the mass of said processed bone.

42. The filament as recited in claim 31, wherein said thermoplastic comprises nylon, acrylonitrile butadiene styrene (ABS), polycarbonate, polyetherimide, polymethylmethacrylate (PMMA), acrylic, polyacryletherketones or similar biocompatible thermoplastic.

43. The filament as recited in claim 38, wherein the filament contains a minimum of 1% bone solid by weight.

44. The filament as recited in claim 31, wherein said bone comprises cortical bone powder, granule or fiber.

45. A system for making an implant having osteoconductive properties; said system comprising:
a production station, said production station comprising at least one of volumetric printing, injection molding, machining, sintering, or forming device adapted to use a filament comprising a bone component and a thermoplastic component in a predetermined ratio;
wherein said implant comprises exposed bone in predetermined areas of said implant to improve osteoconductivity after the implant is implanted into a patient.

46. The system as recited in claim 45 wherein said bone component is substantially evenly distributed in said thermoplastic component in said filament before said filament is used to produce said implant.

47. The system as recited in claim 45 wherein said bone component is at least one of sterilized or processed to reduce bioburden in said bone component before it is added to said thermoplastic component.

48. The system as recited in claim 45 wherein said bone component is distributed substantially evenly with said thermoplastic component in predetermined areas of the filament.

49. The system as recited in claim 45 wherein said bone component is distributed substantially evenly with said thermoplastic component substantially throughout said filament.

50. The system as recited in claim 45 wherein said bone component has a particle size of between less than about 1,000 μm.

51. The system as recited in claim 45 wherein said bone component has a particle size of less than about 500 μm.

52. The system as recited in claim 45, wherein said predetermined ratio is on the order of said thermoplastic component being approximately two times a mass of said bone component.

53. The system as recited in claim 45 wherein said bone component comprises mineral bone solid derived from human or animal bone, said bone component treated via thermal, mechanical, or chemical processes to remove blood and lipids to reduce bioburden, leaving solid mineral components.

54. The system as recited in claim 53 wherein said solid mineral components provide thermal stabilization to bone proteins, allowing for said bone proteins to avoid denaturation during extrusion heating.

55. The system as recited in claim 45 wherein said bone component is mechanically processed to create powdered, granular, elongate, or fiber form, with powder or granular forms having particles less than about 1,000 μm in size and a residual moisture content of less than 6% by weight.

56. The system as recited in claim 45 wherein said bone component is mixed with said thermoplastic component in a specific ratio, the specific ratio is determined by mass, where the mass of said thermoplastic component ranges from 10 to 50 times the mass of said bone component.

57. The system as recited in claim 45, wherein said bone component is mixed with said thermoplastic component in a specific ratio, the ratio is determined by mass, where the mass of said thermoplastic ranges from 2 to 100 times the mass of said bone component.

58. The system as recited in claim 45 wherein said bone component comprises cortical bone powder, granule or fiber and is treated via thermal, mechanical, or chemical processes to remove blood and lipids and reduce bioburden, leaving solid mineral components.

59. The system as recited in claim 45 wherein said thermoplastic component comprises nylon, acrylonitrile butadiene styrene (ABS), polycarbonate, polyetherimide, polymethylmethacrylate (PMMA), acrylic, polyacryletherketones or similar biocompatible thermoplastic.

60. The system as recited in claim 45 wherein the extrusion contains a minimum of 1% bone solid by weight.

61. The system as recited in claim 45 wherein said implant is manufactured from a bone-derived thermoplastic extrusion;
said implant incorporating a combination of human or animal bone-derived solid and thermoplastic with dispersal of said human or animal bone-derived solid in said thermoplastic.

62. The system as recited in claim 45 wherein said implant is manufactured utilizing volumetric printing, injection molding, machining, sintering, forming or similar means.

63. The system as recited in claim 45 wherein there is substantially uniform dispersal of the bone component within the thermoplastic component.

64. The system as recited in claim 61 wherein at least a portion of said human or animal bone-derived solid is exposed at the surface of the implant;
the exposed bone-derived solid expressing osteoconductive and/or osteoinductive properties and imparting the properties to the implant.

65. The system as recited in claim 64 wherein said exposed bone-derived solid on specific surfaces is deposited in a controlled manner by mechanical or chemical means for exposure of osteoconductive or osteoinductive elements where biologic response is desired;
the chemical means comprising treatment of bone with acid such as acetic acid, citric acid, ethylenediamine tetraacetic acid, or hydrochloric acid.

66. The system as recited in claim 45 wherein the implant comprises hygroscopic properties allowing for cellular and/or chemical diffusion and/or communication between internal bone-derived solids and an external surface of said implant.

67. The system as recited in claim 45 wherein the implant is process-strengthened utilizing strain hardening, compression annealing, cross-linking, addition of strengthening additive, or similar means in order to accommodate physiological loading without failure.

68. The system as recited in claim 45 wherein the implant possesses variable zones of differing bone content to impart regional mechanical and biological functions such as a diffusion gradient for directed biologic response.

69. The system as recited in claim 45 wherein said system further comprises:
a filament production station for producing at least one filament;
said filament production station comprising:
an extruder having a feed hopper, said hopper being adapted to receive a mixture of bone and thermoplastic in a predetermined ratio, said extruder plasticating said mixture such that said bone is dispersed substantially evenly throughout said thermoplastic, thereby providing said filament for use at said production station.

70. The system as recited in 69 wherein said system further comprises:
a mixing station for producing said mixture of bone and thermoplastic in said predetermined ratio.

71. The system as recited in claim 70 wherein said predetermined ratio of said thermoplastic component is between two to one-hundred times the mass of said bone component.

72. The system as recited in claim 70 wherein said bone comprises a particle size of less than about 1000 μm.

73. The system as recited in claim 45 wherein said production station comprises at least one volumetric or 3D printer.

74. The system as recited in claim 71, wherein said predetermined ratio is selected in response to osteoconductive properties of said implant.

75. The system as recited in claim 73 wherein said at least one volumetric or 3D printer has a plurality of print heads, each of which is adapted to receive a filament having predetermined bone to thermoplastic ratio.

76. The system as recited in claim 69 wherein said implant comprises predefined areas where osteoconductivity is desired, said at least one filament having bone and thermoplastic ratio such that when said printer prints said implant, said bone is located at said predefined areas.

77. The system as recited in claim 75 wherein a plurality of filaments are used with said plurality of print heads, respectively, each of said plurality of filaments have a different bone to thermoplastic ratio, so that predefined areas of said implant also have corresponding different bone to thermoplastic ratio.

78. The system as recited in claim 69, wherein said at least one filament is used in said print head and said implant comprises predefined areas where osteoconductivity is desired, said at least one filament having bone and thermoplastic ratio such that when said print head prints said implant and directs said bone to said predefined areas.

79. A method for making an osteoconductive implant having osteoconductive areas; said method comprising the steps of:
providing a filament comprising bone and thermoplastic in a predetermined ratio, said bone being substantially evenly dispersed in said thermoplastic in at least a portion of said filament;
using said filament to produce the implant such that bone is located at said osteoconductive areas of said implant.

80. The method as recited in claim 79 wherein said using step comprises the step of:
using a volumetric/3D printer or injection mold to print or mold, respectively said implant using said filament.

81. The method as recited in claim 79 wherein said bone in said filament has a bone particle size of less than about 500 micrometers.

82. The method as recited in claim 79 wherein said method further comprises the step of using a filament wherein said predetermined ratio of thermoplastic to bone is selected in response to the osteoconductive properties desired in the implant.

83. The method as recited in claim 81, wherein said predetermined ratio of thermoplastic mass is approximately two times the mass of said bone.

84. The method as recited in claim 81, wherein said predetermined ratio of thermoplastic mass is approximately ten times the mass of said bone.

85. The method as recited in claim 81, wherein said predetermined ratio of thermoplastic mass is approximately fifty times the mass of said bone.

86. The method as recited in claim 81, wherein said predetermined ratio of thermoplastic mass is approximately one hundred times the mass of said bone.

87. The method as recited in claim 79 wherein said method further comprises the steps of:
determining an amount of bone to situate at said osteoconductive areas;
using at least one volumetric/3D printer and said filament to situate at least some of the bone in said filament at said osteoconductive areas.

88. The method as recited in claim 87, wherein said at least one volumetric/3D printer comprises a plurality of print heads, said method comprising the steps of:
using a first filament having a first predetermined ratio of bone to thermoplastic in one of said plurality of print heads;
using a second filament having a second predetermined ratio of bone to thermoplastic in another of said plurality of print heads;
wherein said first and second predetermined ratios are different.

89. The method as recited in claim 87, wherein said method further comprises the steps of:
using a first filament having a first predetermined ratio of bone to thermoplastic in said at least one volumetric/3D printer to print a first portion of said implant;
using a second filament having a second predetermined ratio of bone to thermoplastic in said at least one volumetric/3D printer to print a second portion of said implant;
wherein said first and second predetermined ratios are different.

90. The method as recited in claim 79 wherein said method further comprises the step of:
demineralizing said implant after it is produced in order for the bone to provide thermal protection to osteoinductive bone proteins, thereby avoiding protein denaturation during heating.

91. The method as recited in claim 79 wherein said method further comprises the step of:
selecting a filament that will cause at least a portion of said osteoconductive areas to have a higher bone content than other portions of said implant.

92. The method as recited in claim 79 wherein said method further comprises the step of:
selecting a filament that will cause at least a portion of said osteoconductive areas to have a low bone content than other portions of said implant.

93. The method as recited in claim 79 wherein said method further comprises the step of:
processing said implant to increase a porosity of the implant to facilitate absorbing fluid having nutrients and/or cells that facilitate a healing response.

94. The method as recited in claim 79 wherein said method further comprises the step of:
processing the bone to a predetermined particle size to provide processed bone;
combining a predetermined amount of said processed bone with a predetermined amount of thermoplastic in said predetermined ratio to provide a mixture;
feeding said mixture into an extruder;
forming said filament using said extruder;
using said filament during said using step.

95. The method as recited in claim 79 wherein said implant is processed chemically or mechanically to expose said bone to facilitate osteoconduction.

96. A surgical implant for implanting into a person, said surgical implant being manufactured according to the method of claim 79.

97. A system for creating a filament having a mixture of bone and thermoplastic in a predetermined ratio, said system comprising:
a filament producing station for producing the filament, said station comprising an extruder adapted to receive said mixture and for creating the filament having said bone distributed substantially evenly in said thermoplastic and for extruding and producing said filament in response thereto.

98. The system as recited in claim 97 wherein said bone is distributed substantially evenly with said thermoplastic substantially throughout said filament.

99. The system as recited in claim 97 wherein said bone has a particle size of 1,000 μm or less.

100. The system as recited in claim 97, wherein said predetermined ratio is on the order of said thermoplastic being approximately two times to 100 times a mass of said bone.

101. The system as recited in claim 97, wherein said bone is at least one of sterilized or processed to reduce bioburden in said bone before it is added to said thermoplastic.

102. The system as recited in claim 97, wherein said bone is distributed substantially evenly with said thermoplastic in predetermined areas of the filament.

103. The system as recited in claim 97, wherein said bone is distributed substantially evenly with said thermoplastic substantially throughout the filament.

104. The system as recited in claim 97, wherein said bone has a particle size of less than 1,000 μm.

105. The system as recited in claim 97, wherein said ratio is on the order of said thermoplastic being approximately two times to a hundred times a mass of said bone.

106. The system as recited in claim 97, wherein said bone comprises mineral bone solid derived from human or animal bone, said bone treated via thermal, mechanical, or chemical processes to remove blood and lipids to reduce bioburden, leaving solid mineral components.

107. The system as recited in claim 106, wherein said solid mineral components provide thermal stabilization to bone proteins, allowing for said bone proteins to avoid denaturation during extrusion heating.

108. The system as recited in claim 97, wherein said bone is mechanically processed to create powdered, granular, elongate, or fiber form, with powder or granular forms having particles less than 1,000 μm in size, residual moisture content less than 6% by weight.

109. The system as recited in claim 97, wherein said bone is mixed with said thermoplastic in a specific ratio, the ratio is determined by mass, where the mass of said thermoplastic ranges from 2 to 100 times the mass of said bone.

110. The system as recited in claim 97, wherein said bone comprises cortical bone powder, granule or fiber and is treated via thermal, mechanical, or chemical processes to remove blood and lipids and reduce bioburden, leaving solid mineral components.

111. The system as recited in claim 97, wherein said thermoplastic comprises nylon, acrylonitrile butadiene styrene (ABS), polycarbonate, polyetherimide, polymethylmethacrylate (PMMA), acrylic, polyacryletherketones or similar biocompatible thermoplastic.

112. The system as recited in claim 107, wherein the extrusion contains a minimum of 1% bone solid by weight.
